(19) United States
(12) Patent Application Publication
(10) Pub. No.: US 2025/0121112 A1
(43) Pub. Date: Apr. 17, 2025

(54) SPACE PURIFICATION DEVICE

(71) Applicant: Panasonic Intellectual Property Management Co., Ltd., Osaka (JP)

(72) Inventors: Toshikazu OONO, Gifu (JP); Yasumi MIYAZAWA, Osaka (JP); Shunichiro OKABE, Kanagawa (JP); Kazuma MATSUMOTO, Aichi (JP); Fumiaki ASAI, Aichi (JP)

(73) Assignee: Panasonic Intellectual Property Management Co., Ltd., Osaka (JP)

(21) Appl. No.: 18/728,511

(22) PCT Filed: Dec. 6, 2022
(86) PCT No.: PCT/JP2022/044892

§ 371 Date: Jul. 12, 2024

(30) Foreign Application Priority Data
Jan. 28, 2022 (JP) 2022-011394

(51) Int. Cl. A61L 9/14; A61L 101/06; A61L 101/20; C02F 1/461

(52) U.S. Cl. CPC A61L 9/145; C02F 1/46109; A61L 2101/06; A61L 2101/20; A61L 2209/11; A61L 2209/13; A61L 2209/213; C02F 2201/46125; C02F 2201/46145; C02F 2201/4615; C02F 2209/29

(57) ABSTRACT

A space purification device according to the present disclosure includes: electrolysis bath; water supply unit; electrode unit (140); first calculator (510) that calculates an increased concentration of hypochlorous acid water per unit time based on (i) a first sodium chloride concentration that is a sodium chloride concentration in the electrolysis bath after the sodium chloride concentration has changed due to an input of the electrolysis accelerator into the electrolysis bath and (ii) hypochlorous acid generation efficiency per unit time set in advance; second calculator (512) that calculates, based on a target hypochlorous acid water concentration in the electrolysis bath, a first hypochlorous acid water concentration that is a hypochlorous acid water concentration in the electrolysis bath before energization by electrode unit (140), and the increased concentration of hypochlorous acid water per unit time calculated by first calculator (510), a required energization time for achieving the target hypochlorous acid water concentration; and electrode controller (540) that performs energization in electrode unit (140) for the required energization time calculated by second calculator (512).

### TECHNICAL FIELD

The present disclosure relates to a space purification device.

### BACKGROUND ART

In order to remove bacteria, fungi, viruses, odors, and the like in the air, a space purification device that generates hypochlorous acid water by electrolysis and releases the generated hypochlorous acid water is known. In a conventional space purification device, an energization time for performing energization for electrolysis and a non-energization time for not performing energization are set as one cycle, and this one cycle is repeated to generate hypochlorous acid water (see PTL 1).

### CITATION LIST

### Patent Literature

PTL 1: Unexamined Japanese Patent Publication No. 2019-30396

### SUMMARY OF THE INVENTION

The conventional space purification device includes a cycle counting unit that counts the number of times the one cycle has been repeated, and determines the energization time, the non-energization time, and a power amount in the energization time based on an increase in the count by the cycle counting unit. However, in the conventional control, there is a possibility that a hypochlorous acid water concentration after the energization time elapses does not necessarily reach a target hypochlorous acid water concentration.

An object of the present disclosure is to provide a space purification device that determines an energization time so that a hypochlorous acid water concentration after an energization time elapses becomes a target hypochlorous acid water concentration.

A space purification device according to the present disclosure includes: an electrolysis bath that mixes an electrolysis accelerator and water; a water supply unit that supplies the water to the electrolysis bath; an electrode unit that generates hypochlorous acid water from the electrolysis accelerator and the water mixed in the electrolysis bath; a first calculator that calculates an increased concentration of hypochlorous acid water per unit time based on (i) a first sodium chloride concentration that is a sodium chloride concentration in the electrolysis bath after the sodium chloride concentration has changed due to an input of the electrolysis accelerator into the electrolysis bath and (ii) hypochlorous acid generation efficiency per unit time set in advance; a second calculator that calculates, based on a target hypochlorous acid water concentration in the electrolysis bath, a first hypochlorous acid water concentration that is a hypochlorous acid water concentration in the electrolysis bath before energization by the electrode unit, and the increased concentration of hypochlorous acid water per unit time calculated by the first calculator, a required energization time for achieving the target hypochlorous acid water concentration; and an electrode controller that performs energization in the electrode unit for the required energization time calculated by the second calculator, thereby achieving the desired object.

According to the present disclosure, it is possible to provide a space purification device that determines an energization time so that a hypochlorous acid water concentration after an energization time elapses becomes a target hypochlorous acid water concentration.

### BRIEF DESCRIPTION OF THE DRAWINGS

FIG. 1 is a diagram illustrating an internal configuration of a space purification device according to a first exemplary embodiment.

FIG. 2 is a schematic functional block diagram of a controller according to the first exemplary embodiment.

FIG. 3 is a flowchart illustrating a control procedure by the controller according to the first exemplary embodiment.

FIG. 4 is a diagram illustrating an internal configuration of a space purification device according to a second exemplary embodiment.

FIG. 5 is a schematic functional block diagram of a controller according to the second exemplary embodiment.

FIG. 6 is a diagram illustrating a data structure of a hypochlorous acid consumption amount table.

FIG. 7 is a flowchart illustrating a control procedure performed by the controller according to the second exemplary embodiment.

FIG. 8 is a schematic functional block diagram of a controller according to a third exemplary embodiment.

FIG. 9 is a schematic functional block diagram of a controller according to a fourth exemplary embodiment.

### DESCRIPTION OF EMBODIMENT

Exemplary embodiments of the present disclosure will be described with reference to the drawings. However, the following exemplary embodiments are exemplified to embody the technical idea of the present disclosure, and the present disclosure is not limited to the following exemplary embodiments. In particular, unless otherwise specified, numerical values, materials, shapes, relative arrangements, and the like described in the exemplary embodiments are not intended to limit the scope of the present disclosure only thereto, and are merely examples.

### First Exemplary Embodiment

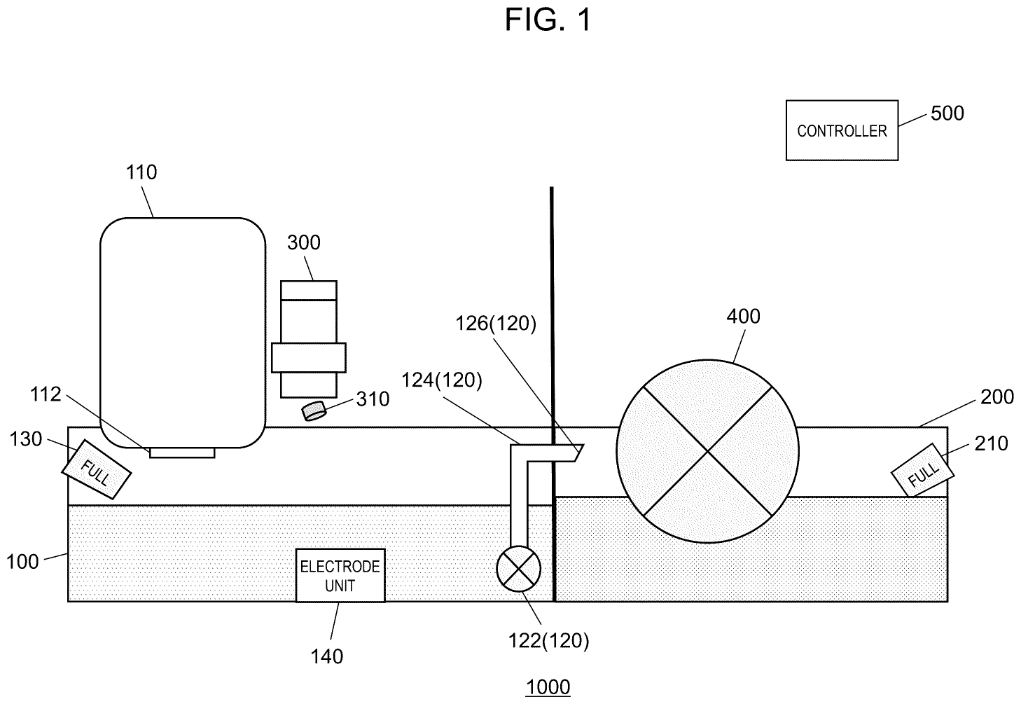

First, space purification device 1000 according to an exemplary embodiment of the present disclosure will be described. FIG. 1 is a diagram illustrating an internal configuration of space purification device 1000.

Space purification device 1000 includes electrolysis bath 100, water supply unit 110, electrolysis accelerator input unit 300, electrode unit 140, electrolytic water supply unit 120, purification bath 200, electrolysis bath full water float 130, purification bath full water float 210, and controller 500.

Electrolysis bath 100 has a box shape with a top surface opened, has a structure capable of storing water, and stores water supplied from water supply unit 110 described later. Electrolysis bath 100 is disposed, for example, in a lower portion of space purification device 1000.

Water supply unit 110 is a tank that stores water therein, and is detachable from electrolysis bath 100. Lid 112 is provided in an opening (not illustrated) of water supply unit 110, and an opening and closing part (not illustrated) is provided at a center of lid 112. When the opening and closing part is opened, water in water supply unit 110 is supplied to electrolysis bath 100. Specifically, when water supply unit 110 is attached to electrolysis bath 100 with the opening of water supply unit 110 facing downward, the opening and closing part is opened. That is, when water supply unit 110 containing water is attached to electrolysis bath 100, the opening and closing part is opened, water is supplied to electrolysis bath 100, and the water is stored in electrolysis bath 100. When a water level of electrolysis bath 100 increases and reaches a position of lid 112, the water supply to electrolysis bath 100 is stopped because the opening of water supply unit 110 is water-sealed. In a case where water remains in water supply unit 110, the water in water supply unit 110 is supplied to electrolysis bath 100 every time the water level in electrolysis bath 100 drops. As a result, the water level in electrolysis bath 100 is kept constant. Note that water supply unit 110 may not be a tank that stores water. In this case, tap water is used to supply water to electrolysis bath 100. Then, in a case where the water level in electrolysis bath 100 drops, tap water may be supplied until the water level in electrolysis bath 100 increases to a predetermined position.

Electrolysis accelerator input unit 300 is disposed in an upper portion of electrolysis bath 100. Electrolysis accelerator input unit 300 can load electrolysis accelerator 310 therein, and rotates a tablet inputting member (not illustrated) when receiving an instruction to input electrolysis accelerator 310 from controller 500. When the tablet inputting member rotates, electrolysis accelerator 310 falls into electrolysis bath 100. Electrolysis accelerator input unit 300 counts the number of electrolysis accelerators 310 dropped into electrolysis bath 100, and stops the rotation of the tablet inputting member when determining that one tablet of electrolysis accelerator 310 has fallen into electrolysis bath 100. That is, electrolysis accelerator input unit 300 inputs electrolysis accelerator 310 into electrolysis bath 100. Electrolysis accelerator 310 is sodium chloride and is formed as an electrolysis accelerating tablet. By dissolving electrolysis accelerator 310 in water in electrolysis bath 100, water containing chloride ions is generated in electrolysis bath 100. That is, electrolysis bath 100 mixes electrolysis accelerator 310 and water.

Electrode unit 140 is installed so as to be immersed in water in electrolysis bath 100. When energized, electrode unit 140 electrochemically electrolyzes water containing chloride ions in electrolysis bath 100 to generate hypochlorous acid water (electrolytic water).

Electrolytic water supply unit 120 includes water supply pump 122 and water supply pipe 124.

Water supply pump 122 is disposed in electrolysis bath 100, and is connected to water supply pipe 124. Water supply pump 122 operates in response to an instruction from controller 500 to pump hypochlorous acid water generated in electrolysis bath 100 toward water supply pipe 124.

Water supply pipe 124 is a pipe connecting electrolysis bath 100 and purification bath 200, and includes supply port 126 on a side of purification bath 200. The hypochlorous acid water pumped up by water supply pump 122 flows in water supply pipe 124 and is supplied from supply port 126 to purification bath 200. That is, water supply pump 122, water supply pipe 124, and supply port 126 supply the hypochlorous acid water from electrolysis bath 100 to purification bath 200.

Purification bath 200 has a box shape with a top surface opened, and stores the hypochlorous acid water supplied from electrolysis bath 100 by electrolytic water supply unit 120. That is, the hypochlorous acid water generated in electrolysis bath 100 is stored. Purification bath 200 is provided with purification unit 400.

Purification unit 400 includes a fan and a filter. The fan is, for example, a sirocco fan, and rotates under the control of controller 500. As the fan rotates, air is sucked into space purification device 1000 from an intake port provided in a housing of space purification device 1000. The filter is a member that brings the hypochlorous acid water stored in purification bath 200 into contact with the indoor air flowing into space purification device 1000 by the fan. The filter is formed in a cylindrical shape, and holes through which air can flow are provided in a circumferential portion. The filter is built in purification bath 200 so that one end of the filter can be immersed in the hypochlorous acid water stored in purification bath 200 to retain water. Furthermore, the filter is built in purification bath 200 in a configuration capable of rotating about a central axis as a rotation center. The filter is rotated by a drive unit to bring the hypochlorous acid water and the indoor air into continuous contact with each other.

Incidentally, an air path leading from the intake port to the filter, the fan, and a blow-out port is formed inside space purification device 1000. When the fan rotates, external air sucked from the intake port and entering the air path is sequentially blown out to the outside of space purification device 1000 via the filter, the fan, and the blow-out port in this order. As a result, a gas containing the hypochlorous acid water in purification bath 200 is released to the outside. That is, purification unit 400 purifies a space using the hypochlorous acid water stored in purification bath 200.

Moreover, electrolysis bath 100 includes electrolysis bath full water float 130. Purification bath 200 includes purification bath full water float 210. Electrolysis bath full water float 130 and purification bath full water float 210 detect whether or not water or hypochlorous acid water is present. Here, water, hypochlorous acid water, and mixed water of the water and the hypochlorous acid water are collectively referred to as “water”. Electrolysis bath full water float 130 and purification bath full water float 210 are collectively referred to as “floats”.

Each float has buoyancy and is further provided with a magnet (not illustrated), and a position of the magnet is detected by a detection part (not illustrated). In a case where water is present to a position of each float, each float moves to a predetermined position by buoyancy. The detection part detects a magnet provided in each float portion. On the other hand, in a case where there is no water up to the position of each float, the detection part cannot detect the magnet provided in each float.

Electrolysis bath full water float 130 detects the full water of electrolysis bath 100. Purification bath full water float 210 detects the full water of purification bath 200. Here, the full water does not have to be an amount of water (position) of 100% with respect to the capacity of electrolysis bath 100 or purification bath 200, and an amount of water that does not overflow even if water is further charged may be the full water. Each float transmits a detection result to controller 500.

Controller 500 receives detection results from electrolysis bath full water float 130 and purification bath full water float 210. Furthermore, controller 500 performs controls of electrolytic water supply unit 120, electrode unit 140, electrolysis accelerator input unit 300, and purification unit 400. Details of processing of controller 500 will be described later.

Here, an example of a flow from generation to release of hypochlorous acid water will be described.

Firstly, electrolysis bath 100 and purification bath 200 are made to a state of free of water. This corresponds to a state in which there is no water, for example, in a case where space purification device 1000 is installed after purchase of space purification device 1000, or after maintenance such as drainage of water and cleaning of electrolysis bath 100 and purification bath 200.

A user injects water into water supply unit 110, and attaches water supply unit 110 to electrolysis bath 100. When water supply unit 110 is attached to electrolysis bath 100, the opening and closing part of lid 112 is opened, whereby water is supplied from water supply unit 110 to electrolysis bath 100. Water is supplied until the opening of water supply unit 110 is water-sealed. A state in which the opening of water supply unit 110 is water-sealed is set to a state of full water. Note that tap water may be supplied until the water level in electrolysis bath 100 becomes the state of full water. Whether or not electrolysis bath 100 is in the state of full water can be determined by a detection result of electrolysis bath full water float 130.

Electrolysis accelerator input unit 300 drops electrolysis accelerator 310 toward electrolysis bath 100, and submerged electrolysis accelerator 310 is dissolved in water. As a result, electrolysis bath 100 becomes the state of full water with water containing chloride ions. Note that the user may drop electrolysis accelerator 310.

By energizing electrode unit 140, controller 500 electrolyzes water containing chloride ions to generate hypochlorous acid water. At this time, energization is performed in electrode unit 140 for a required energization time for achieving a target hypochlorous acid water concentration. As a result, the hypochlorous acid water having the target hypochlorous acid water concentration is generated. A method of calculating the required energization time for achieving the target hypochlorous acid water concentration will be described later.

When the hypochlorous acid water having the target hypochlorous acid water concentration is generated, controller 500 supplies the hypochlorous acid water having the target hypochlorous acid water concentration to purification bath 200 by operating water supply pump 122. As a result, purification bath 200 becomes the state of full water. Controller 500 operates purification unit 400 to release air brought into contact with the hypochlorous acid water in purification bath 200 to the outside of space purification device 1000. By releasing the air brought into contact with the hypochlorous acid water in purification bath 200 to the outside of space purification device 1000, a water level of the hypochlorous acid water in purification bath 200 decreases with the lapse of time.

When water supply pump 122 is operated, a water level of hypochlorous acid water in electrolysis bath 100 decreases. However, when the opening and closing part of lid 112 of water supply unit 110 is opened, water is supplied from water supply unit 110 to electrolysis bath 100, and the water level of electrolysis bath 100 is kept constant.

When the water is supplied from water supply unit 110 to electrolysis bath 100, hypochlorous acid water in electrolysis bath 100 is diluted with the water supplied from water supply unit 110. As a result, a hypochlorous acid water concentration in electrolysis bath 100 becomes lower than the target hypochlorous acid water concentration. Note that, in a case where the water level in electrolysis bath 100 decreases, tap water may be supplied until the water level in electrolysis bath 100 becomes the state of full water.

When the hypochlorous acid water concentration in electrolysis bath 100 becomes lower than the target hypochlorous acid water concentration, controller 500 executes energization to electrode unit 140. As a result, the hypochlorous acid water concentration in electrolysis bath 100 becomes the target hypochlorous acid water concentration again. A method of calculating the energization time will be described later.

Incidentally, as described above, since the water level of the hypochlorous acid water in purification bath 200 decreases with the lapse of time, the second hypochlorous acid water supply is performed after the lapse of a predetermined time from the first hypochlorous acid water supply to purification bath 200. As a result, space purification device 1000 can continue releasing a gas containing the hypochlorous acid water.

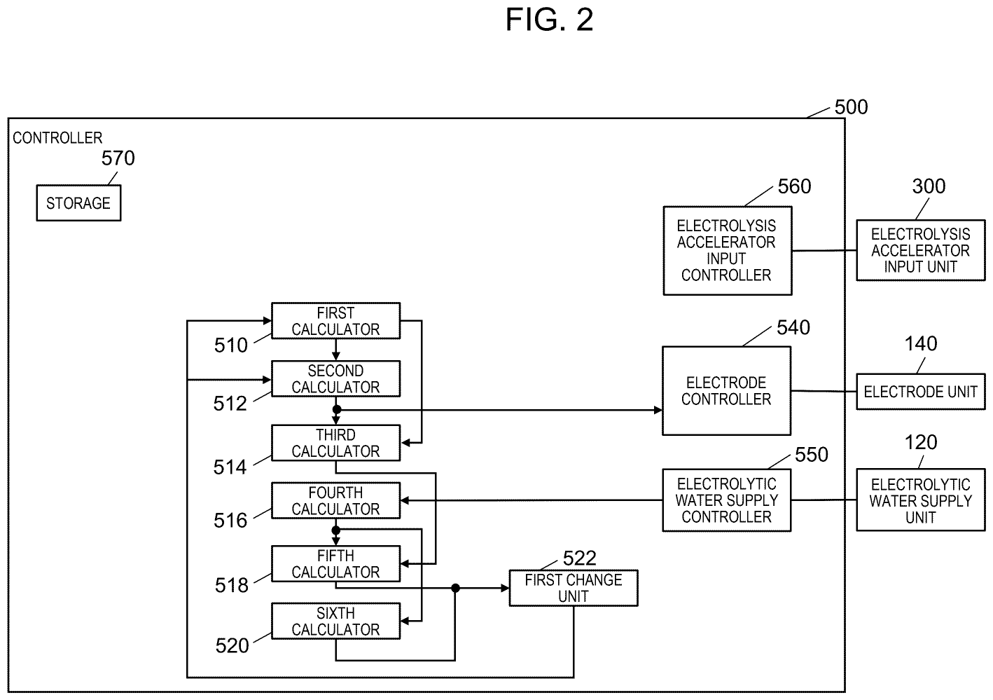

Next, each function and a control flow of controller 500 according to the present exemplary embodiment will be described with reference to FIG. 2. FIG. 2 is a schematic functional block diagram of controller 500 according to the present exemplary embodiment.

Controller 500 includes electrolysis accelerator input controller 560, first calculator 510, storage 570, second calculator 512, electrode controller 540, third calculator 514, electrolytic water supply controller 550, fourth calculator 516, fifth calculator 518, sixth calculator 520, and first change unit 522.

Electrolysis accelerator input controller 560 controls an input of electrolysis accelerator 310 by electrolysis accelerator input unit 300.

First calculator 510 calculates an increased concentration of hypochlorous acid water per unit time based on a first sodium chloride concentration that is a sodium chloride concentration in electrolysis bath 100 after the sodium chloride concentration is changed due to an input of electrolysis accelerator 310 into electrolysis bath 100, and hypochlorous acid generation efficiency per unit time set in advance. The input of electrolysis accelerator 310 into electrolysis bath 100 may be performed by electrolysis accelerator input unit 300 according to an instruction from electrolysis accelerator input controller 560, or may be performed by a user. Hereinafter, a specific description will be given using numerical values, but the numerical values are merely examples, and other numerical values may be used.

Firstly, first calculator 510 calculates the first sodium chloride concentration that is a sodium chloride concentration in electrolysis bath 100 after the sodium chloride concentration changes due to the input of electrolysis accelerator 310 into electrolysis bath 100. In a case where water is supplied from water supply unit 110 to electrolysis bath 100 from a state where there is no water in electrolysis bath 100 and purification bath 200 to be a state of full water, the first sodium chloride concentration is 0 [mg/L (liter)]. Assuming that a capacity of electrolysis bath 100 is 1 [L] and a weight of electrolysis accelerator 310 is 500 [mg], the calculation formula is as follows: first sodium chloride concentration after sodium chloride concentration changes due to input of electrolysis accelerator 310 into electrolysis bath 100=weight of electrolysis accelerator 310/capacity of electrolysis bath 100=500 [mg/L]/1 [L]=500 [mg/L]. The capacity of electrolysis bath 100 and the weight of electrolysis accelerator 310 are stored in advance in storage 570. Storage 570 is a so-called memory, and stores various values.

Next, first calculator 510 acquires hypochlorous acid generation efficiency per unit time set in advance from storage 570. The hypochlorous acid generation efficiency per unit time is a value determined in advance by an experiment or the like, and can be arbitrarily set. The hypochlorous acid generation efficiency per unit time varies depending on a type of an electrode of electrode unit 140, a size of the electrode, a current value flowing through the electrodes, and the like. In the present exemplary embodiment, as an example, the hypochlorous acid generation efficiency per unit time is set to 1 [%/min](min: minute).

Next, first calculator 510 calculates an increased concentration of hypochlorous acid water per unit time. The calculation formula is as follows: increased concentration of hypochlorous acid water per unit time=first sodium chloride concentration×hypochlorous acid generation efficiency per unit time=500 [mg/L]×1 [%/min]=5 [mg/(L×min)].

Second calculator 512 calculates a required energization time for achieving a target hypochlorous acid water concentration based on the target hypochlorous acid water concentration in electrolysis bath 100, a first hypochlorous acid water concentration that is a hypochlorous acid water concentration in electrolysis bath 100 before energization by electrode unit 140, and the increased concentration of hypochlorous acid water per unit time calculated by first calculator 510.

Specifically, first of all, second calculator 512 acquires the target hypochlorous acid water concentration in electrolysis bath 100 from storage 570. In the present exemplary embodiment, the target hypochlorous acid water concentration is set to 10 [ppm](ppm: parts per million). Furthermore, the target hypochlorous acid water concentration may be changeable by the user. For example, the user may set the target hypochlorous acid water concentration in a remote controller (not illustrated) or the like, and the controller 500 may acquire the set target hypochlorous acid water concentration and rewrite the target hypochlorous acid water concentration in storage 570 to the target hypochlorous acid water concentration set by the user.

Next, second calculator 512 determines the first hypochlorous acid water concentration that is a hypochlorous acid water concentration in electrolysis bath 100 before energization by electrode unit 140. At the first time, since electrolysis bath 100 is in the state of full water and hypochlorous acid does not exist in the water in electrolysis bath 100, second calculator 512 determines 0 [ppm] as the first hypochlorous acid water concentration.

Next, second calculator 512 calculates a required energization time for achieving the target hypochlorous acid water concentration. The calculation formula is as follows: required energization time=(target hypochlorous acid water concentration−hypochlorous acid water concentration before energization)/increased concentration of hypochlorous acid water per unit time calculated by first calculator 510=(10 [ppm]−0 [ppm])/5 [mg/(L×min)]=2 [min].

Electrode controller 540 performs energization in electrode unit 140 for the required energization time calculated by second calculator 512. Specifically, electrode controller 540 performs energization for 2 [min] that is the required energization time calculated by second calculator 512. Thus, the hypochlorous acid water having the target hypochlorous acid water concentration can be generated in electrolysis bath 100.

Third calculator 514 calculates a second sodium chloride concentration that is a sodium chloride concentration in electrolysis bath 100 after energization by electrode unit 140 based on the first sodium chloride concentration, the increased concentration of hypochlorous acid water per unit time calculated by first calculator 510, and the required energization time calculated by second calculator 512. The sodium chloride concentration in electrolysis bath 100 after energization is lower than the sodium chloride concentration in electrolysis bath 100 before energization.

Specifically, first of all, third calculator 514 acquires the first sodium chloride concentration, the increased concentration of hypochlorous acid water per unit time, and the required energization time. In the present exemplary embodiment, the first sodium chloride concentration=500 [mg/L], the increased concentration of hypochlorous acid water per unit time=5 [mg/(L×min)], and the required energization time=2 [min] are acquired.

Next, third calculator 514 calculates the second sodium chloride concentration. The calculation formula is as follows: second sodium chloride concentration=first sodium chloride concentration−(increased concentration of hypochlorous acid water per unit time×required energization time)=500 [mg/L]−(5 [mg/(L×min)]×2 [min])=490 [mg/L]. As a result, the sodium chloride concentration in electrolysis bath 100 after energization by electrode unit 140 can be grasped.

Electrolytic water supply controller 550 supplies hypochlorous acid water to purification bath 200 by electrolytic water supply unit 120. Specifically, the hypochlorous acid water is supplied by electrolytic water supply unit 120 until purification bath 200 becomes the state of full water. Whether or not purification bath 200 is in the state of full water can be determined based on a detection result of purification bath full water float 210.

Fourth calculator 516 calculates an amount of hypochlorous acid water supplied by electrolytic water supply unit 120. In the present exemplary embodiment, as an example, it is assumed that water supply time during which the water is supplied by electrolytic water supply unit 120 until purification bath 200 becomes the state of full water is 5 [s (sec)]. Furthermore, a water supply amount per unit time is set to 50 [mL/s]. The water supply amount per unit time is a value determined in advance by an experiment or the like, and varies depending on a water supply capacity of electrolytic water supply unit 120. The water supply amount per unit time is stored in storage 570.

Fourth calculator 516 calculates the hypochlorous acid water supply amount. The calculation formula is as follows: hypochlorous acid water supply amount=water supply amount per unit time×water supply time=50 [mL/s]×5 [s]=250 [mL].

Fifth calculator 518 calculates a third sodium chloride concentration that is a sodium chloride concentration in electrolysis bath 100 after the supply of electrolytic water by electrolytic water supply unit 120 based on the second sodium chloride concentration calculated by third calculator 514, the hypochlorous acid water supply amount calculated by fourth calculator 516, and the capacity of electrolysis bath 100. The sodium chloride concentration in electrolysis bath 100 after the supply of the electrolytic water is lower than the sodium chloride concentration in electrolysis bath 100 before the supply of the electrolytic water due to water supplied by water supply unit 110.

Specifically, first of all, fifth calculator 518 acquires the second sodium chloride concentration calculated by third calculator 514, the hypochlorous acid water supply amount calculated by fourth calculator 516, and the capacity of electrolysis bath 100. In the present exemplary embodiment, fifth calculator 518 acquires the second sodium chloride concentration=490 [mg/L] calculated by third calculator 514, the hypochlorous acid water supply amount=250 [mL] calculated by fourth calculator 516, and the capacity of electrolysis bath=1 [L].

Next, fifth calculator 518 calculates the third sodium chloride concentration. The calculation formula is as follows: third sodium chloride concentration=second sodium chloride concentration−(second sodium chloride concentration×(hypochlorous acid water supply amount/capacity of electrolysis bath 100)=490 [mg/L]−(490 [mg/L]×(250 [mL]/1000 [mL]))=367.5 [mg/L]. As a result, it is possible to grasp the sodium chloride concentration in electrolysis bath 100 after the supply of the electrolytic water by electrolytic water supply unit 120.

Sixth calculator 520 calculates a second hypochlorous acid water concentration that is a hypochlorous acid water concentration in electrolysis bath 100 after the supply of the hypochlorous acid water by electrolytic water supply unit 120 based on the target hypochlorous acid water concentration, the hypochlorous acid water supply amount calculated by fourth calculator 516, and the capacity of electrolysis bath 100. The hypochlorous acid water concentration in electrolysis bath 100 after the supply of the hypochlorous acid water is lower than the hypochlorous acid water concentration (target hypochlorous acid water concentration) in electrolysis bath 100 before the supply of the hypochlorous acid water due to the water supplied by water supply unit 110.

Specifically, first of all, sixth calculator 520 acquires the target hypochlorous acid water concentration, the hypochlorous acid water supply amount calculated by fourth calculator 516, and the capacity of electrolysis bath 100. In the present exemplary embodiment, sixth calculator 520 acquires the target hypochlorous acid water concentration=10 [ppm], the hypochlorous acid water supply amount=250 [mL] calculated by fourth calculator 516, and the capacity 1 [L] of electrolysis bath 100.

Next, sixth calculator 520 calculates the second hypochlorous acid water concentration. The calculation formula is as follows: second hypochlorous acid water concentration=target hypochlorous acid water concentration−(target hypochlorous acid water concentration×(hypochlorous acid water supply amount/capacity of electrolysis bath 100)=10 [ppm]−(10 [ppm]×(250 [mL]/1000 [mL]))=7.5 [ppm]. As a result, the hypochlorous acid water concentration in electrolysis bath 100 after the supply of the hypochlorous acid water can be grasped.

First change unit 522 changes the first sodium chloride concentration used for the calculation by first calculator 510 to the third sodium chloride concentration calculated by fifth calculator 518. Furthermore, first change unit 522 changes the first hypochlorous acid water concentration used for the calculation by second calculator 512 to the second hypochlorous acid water concentration calculated by sixth calculator 520.

Specifically, first of all, first change unit 522 acquires the first sodium chloride concentration before the change and the first hypochlorous acid water concentration before the change. The first sodium chloride concentration before the change and the first hypochlorous acid water concentration before the change are stored in storage 570 by, for example, first calculator 510 or second calculator 512. In the present exemplary embodiment, first change unit 522 acquires the first sodium chloride concentration before the change=500 [mg/L] and the first hypochlorous acid water concentration before the change=0 [ppm].

First change unit 522 changes the first sodium chloride concentration before the change=500 [mg/L] to the third sodium chloride concentration=367.5 [mg/L] calculated by fifth calculator 518. Furthermore, first change unit 522 changes the first hypochlorous acid water concentration before the change=0 [ppm] to the second hypochlorous acid water concentration calculated by sixth calculator=7.5 [ppm]. That is, first change unit 522 updates the first sodium chloride concentration to the latest sodium chloride concentration in electrolysis bath 100, and updates the first hypochlorous acid water concentration to the latest hypochlorous acid water concentration in electrolysis bath 100.

First calculator 510 calculates a new increased concentration of hypochlorous acid water per unit time based on a new first sodium chloride concentration (first sodium chloride concentration after the change=the third sodium chloride concentration) changed by first change unit 522 and the hypochlorous acid generation efficiency per unit time set in advance.

Specifically, first of all, first calculator 510 acquires the first sodium chloride concentration after the change and the hypochlorous acid generation efficiency per unit time set in advance. In the present exemplary embodiment, first calculator 510 acquires the first sodium chloride concentration after the change=367.5 [mg/L] and the hypochlorous acid generation efficiency per unit time set in advance=1 [%/min].

Next, first calculator 510 calculates a new increased concentration of hypochlorous acid water per unit time. A specific calculation formula is as follows: new increased concentration of hypochlorous acid water per unit time=first sodium chloride concentration after the change×hypochlorous acid generation efficiency per unit time=367.5 [mg/L]×1 [%/min]=3.675 [mg/(L×min)].

Second calculator 512 calculates a new required energization time by electrode unit 140 based on the target hypochlorous acid water concentration in electrolysis bath 100, the new first hypochlorous acid water concentration (the first hypochlorous acid water concentration after the change=the second hypochlorous acid water concentration) changed by first change unit 522, and the new increased concentration of hypochlorous acid water per unit time calculated by first calculator 510.

Specifically, first of all, second calculator 512 acquires the target hypochlorous acid water concentration in electrolysis bath 100, the first hypochlorous acid water concentration after the change, and the new increased concentration of hypochlorous acid water per unit time calculated by first calculator 510. In the present exemplary embodiment, second calculator 512 acquires the target hypochlorous acid water concentration=10 [ppm] in electrolysis bath 100, the first hypochlorous acid water concentration after the change=7.5 [ppm], and the new increased concentration of hypochlorous acid water per unit time=3.675 [mg/(L×min)] calculated by first calculator 510.

Next, second calculator 512 calculates the new required energization time. A specific calculation formula is as follows: new required energization time=(target hypochlorous acid water concentration−first hypochlorous acid water concentration after the change)/new increased concentration of hypochlorous acid water per unit time calculated by first calculator 510=(10 [ppm]−7.5 [ppm])/3.675 [mg/(L×min)]=0.68 [min].

Electrode controller 540 performs energization in electrode unit 140 for the new required energization time calculated by second calculator 512. In the present exemplary embodiment, second calculator 512 performs energization in electrode unit 140 for the new required energization time=0.68 [min]. Thus, the hypochlorous acid water having the target hypochlorous acid water concentration can be generated again in electrolysis bath 100.

Thereafter, controller 500 repeats the calculation by third calculator 514, the calculation by fourth calculator 516, the calculation by fifth calculator 518, the calculation by sixth calculator 520, the change by first change unit 522, the calculation by first calculator 510, and the calculation by second calculator 512. That is, controller 500 repeats the change of the first sodium chloride concentration and the change of the first hypochlorous acid water concentration by first change unit 522, the calculation of the new increased concentration of hypochlorous acid water per unit time by first calculator 510, the calculation of the new required energization time by second calculator 512, and the control of energizing for the new required energization time by electrode controller 540. As a result, the hypochlorous acid water concentration in electrolysis bath 100 can be continuously set to the target hypochlorous acid water concentration.

Each functional block of controller 500 can be realized by an element such as a central processing unit (CPU) of a computer or a mechanical device in terms of hardware, and is realized by a computer program or the like in terms of software, but here, functional blocks realized by cooperation thereof are illustrated. Therefore, these functional blocks can be implemented in various forms by a combination of hardware and software.

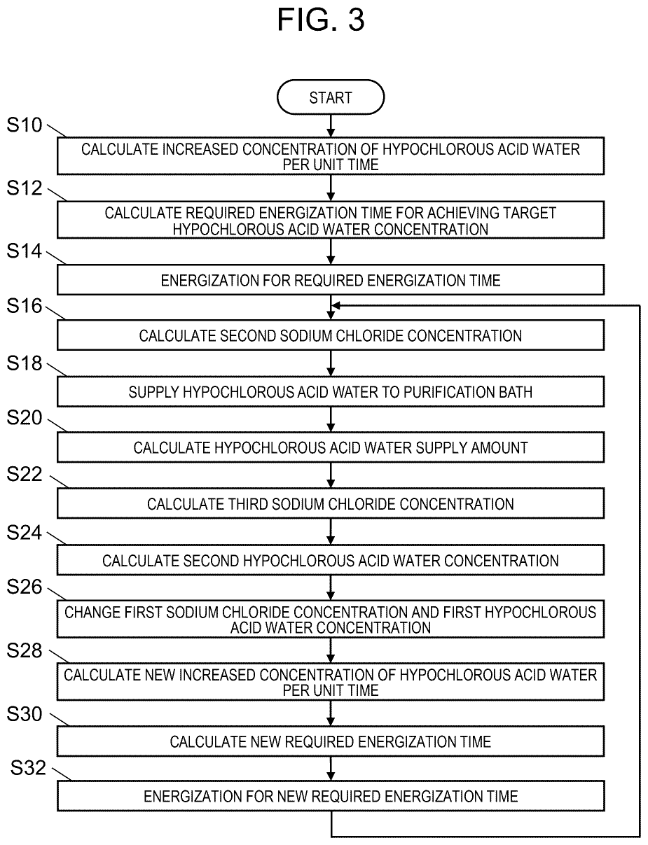

A flowchart of controller 500 having the above configuration will be described. FIG. 3 is a flowchart illustrating control of controller 500 according to the present exemplary embodiment.

Firstly, first calculator 510 calculates an increased concentration of hypochlorous acid water per unit time (S10). Second calculator 512 calculates a required energization time for achieving the target hypochlorous acid water concentration (S12). Electrode controller 540 performs energization in electrode unit 140 for the required energization time calculated by second calculator 512 (S14). Thus, the hypochlorous acid water having the target hypochlorous acid water concentration can be generated in electrolysis bath 100.

Third calculator 514 calculates a second sodium chloride concentration that is a sodium chloride concentration in electrolysis bath 100 after energization by electrode unit 140 (S16). As a result, the sodium chloride concentration in electrolysis bath 100 after energization by electrode unit 140 can be grasped.

Electrolytic water supply controller 550 supplies hypochlorous acid water to purification bath 200 by electrolytic water supply unit 120 until purification bath 200 becomes the state of full water (S18). As a result, the hypochlorous acid water having the target hypochlorous acid water concentration can be supplied to purification bath 200.

Fourth calculator 516 calculates the amount of hypochlorous acid water supplied by electrolytic water supply unit 120 (S20). Fifth calculator 518 calculates a third sodium chloride concentration that is a sodium chloride concentration in electrolysis bath 100 after the supply of the electrolytic water by electrolytic water supply unit 120 (S22). As a result, it is possible to grasp the sodium chloride concentration in electrolysis bath 100 after the supply of the electrolytic water by electrolytic water supply unit 120.

Sixth calculator 520 calculates a second hypochlorous acid water concentration that is a hypochlorous acid water concentration in electrolysis bath 100 after the supply of the hypochlorous acid water by electrolytic water supply unit 120 (S24). As a result, the hypochlorous acid water concentration in electrolysis bath 100 after the supply of the hypochlorous acid water can be grasped.

First change unit 522 changes the first sodium chloride concentration to the third sodium chloride concentration calculated by fifth calculator 518, and changes the first hypochlorous acid water concentration to the second hypochlorous acid water concentration calculated by sixth calculator 520 (S26).

First calculator 510 calculates a new increased concentration of hypochlorous acid water per unit time based on a new first sodium chloride concentration changed by first change unit 522 and hypochlorous acid generation efficiency per unit time set in advance (S28).

Second calculator 512 calculates a new required energization time by electrode unit 140 based on the target hypochlorous acid water concentration in electrolysis bath 100, a new first hypochlorous acid water concentration changed by first change unit 522, and the new increased concentration of hypochlorous acid water per unit time (S30).

Electrode controller 540 performs energization in electrode unit 140 for the new required energization time calculated by second calculator 512 (S32). Thus, the hypochlorous acid water having the target hypochlorous acid water concentration can be generated again in electrolysis bath 100. Thereafter, the process returns to step S16. Controller 500 repeats steps S16 to S32. As a result, the hypochlorous acid water concentration in electrolysis bath 100 can be continuously set to the target hypochlorous acid water concentration.

### Second Exemplary Embodiment

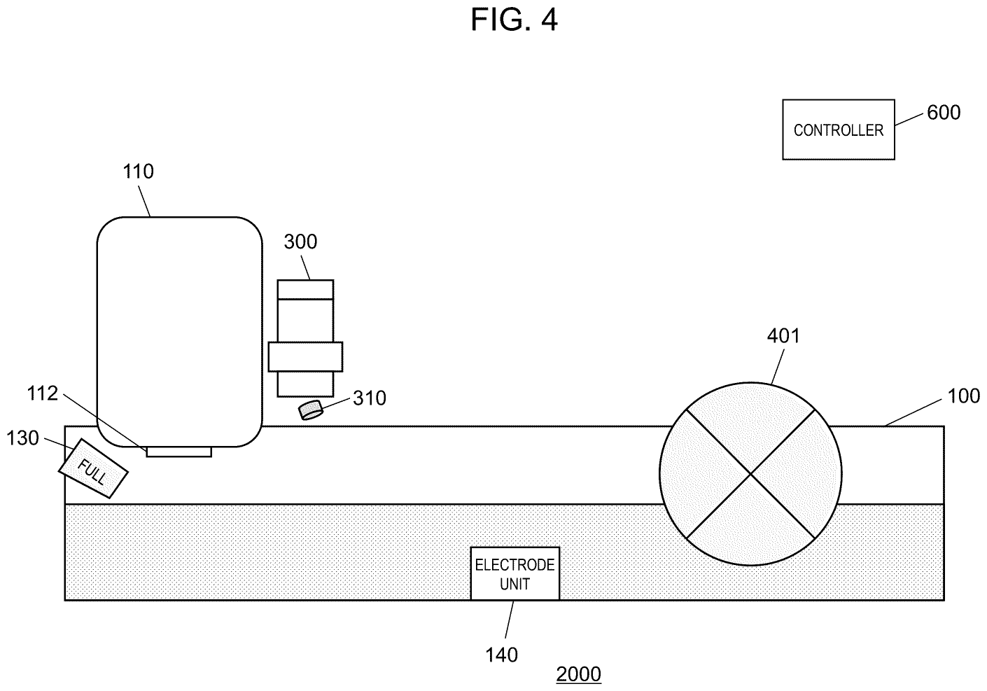

In a second exemplary embodiment, differences from the first exemplary embodiment will be mainly described with reference to FIG. 4. FIG. 4 is a diagram illustrating an internal configuration of space purification device 2000 in the second exemplary embodiment. Space purification device 2000 is partially different in structure from space purification device 1000. A difference between space purification device 2000 and space purification device 1000 will be described.

Space purification device 2000 does not include electrolytic water supply unit 120, purification bath 200, and purification bath full water float 210 of space purification device 1000, and controller 500 is changed to controller 600. Moreover, in space purification device 1000, purification unit 400 is provided in purification bath 200, but in space purification device 2000, purification unit 401 is provided in electrolysis bath 100. That is, space purification device 2000 includes electrolysis bath 100, water supply unit 110, electrolysis accelerator input unit 300, electrode unit 140, electrolysis bath full water float 130, and controller 600.

Electrolysis bath 100, water supply unit 110, electrolysis accelerator input unit 300, and electrode unit 140 have configurations similar to those of the first exemplary embodiment. However, electrolysis accelerator input unit 300 is different in that a tablet inputting member is rotated when an instruction to input electrolysis accelerator 310 is given from controller 600 instead of controller 500. Electrolysis bath 100 is provided with purification unit 401.

Purification unit 401 includes a fan and a filter. The fan rotates under the control of controller 600. As the fan rotates, air is sucked into space purification device 2000 from an intake port provided in a housing of space purification device 2000. The filter is a member that brings hypochlorous acid water stored in electrolysis bath 100 into contact with indoor air flowing into space purification device 2000 by the fan. The filter is formed in a cylindrical shape, and holes through which air can flow are provided in a circumferential portion. The filter is built in electrolysis bath 100 so that one end of the filter can be immersed in the hypochlorous acid water stored in electrolysis bath 100 to retain water. Furthermore, the filter is built in electrolysis bath 100 in a configuration capable of rotating about a central axis as a rotation center. The filter is rotated by a drive unit to bring the hypochlorous acid water and the indoor air into continuous contact with each other.

Incidentally, an air path leading from the intake port to the filter, the fan, and a blow-out port is formed inside space purification device 2000. When the fan rotates, external air sucked from the intake port and entering the air path is sequentially blown out to the outside of space purification device 2000 via the filter, the fan, and the blow-out port in this order. As a result, a gas containing the hypochlorous acid water in electrolysis bath 100 is released to the outside. That is, purification unit 401 purifies a space using the hypochlorous acid water stored in electrolysis bath 100.

Electrolysis bath full water float 130 provided in electrolysis bath 100 is similar to in the first exemplary embodiment. Electrolysis bath full water float 130 transmits a detection result to controller 600.

Controller 600 receives the detection result from electrolysis bath full water float 130. Furthermore, controller 600 performs controls of electrode unit 140, electrolysis accelerator input unit 300, and purification unit 401. Details of processing of controller 600 will be described later.

Here, an example of a flow from generation to release of hypochlorous acid water in the second exemplary embodiment will be described.

Firstly, electrolysis bath 100 is made to a state of free of water. This corresponds to a state where there is no water, for example, in a case where space purification device 2000 is installed after purchase of space purification device 2000 or after maintenance such as drainage of water and cleaning of electrolysis bath 100.

A user injects water into water supply unit 110, and attaches water supply unit 110 to electrolysis bath 100. When water supply unit 110 is attached to electrolysis bath 100, water is supplied from water supply unit 110 to electrolysis bath 100. Water is supplied until the opening of water supply unit 110 is water-sealed. A state in which the opening of water supply unit 110 is water-sealed is set to a state of full water. Note that tap water may be supplied until the water level in electrolysis bath 100 becomes the state of full water.

Electrolysis accelerator input unit 300 drops electrolysis accelerator 310 toward electrolysis bath 100, and submerged electrolysis accelerator 310 is dissolved in water. As a result, electrolysis bath 100 becomes the state of full water with water containing chloride ions. Note that the user may drop electrolysis accelerator 310.

By energizing electrode unit 140, controller 600 electrolyzes water containing chloride ions to generate hypochlorous acid water. At this time, energization is performed in electrode unit 140 for a required energization time for achieving a target hypochlorous acid water concentration. As a result, the hypochlorous acid water having the target hypochlorous acid water concentration is generated.

Controller 600 operates purification unit 401 to release air brought into contact with the hypochlorous acid water in electrolysis bath 100 to the outside of space purification device 2000. By releasing the air brought into contact with the hypochlorous acid water in electrolysis bath 100 to the outside of space purification device 2000, a water level of the hypochlorous acid water in electrolysis bath 100 decreases with the lapse of time. However, when the opening and closing part of lid 112 of water supply unit 110 is opened, water is supplied from water supply unit 110 to electrolysis bath 100, and the water level of electrolysis bath 100 is kept constant.

However, when water is supplied from water supply unit 110 to electrolysis bath 100, the hypochlorous acid water in electrolysis bath 100 is diluted by the water supplied from water supply unit 110, and a hypochlorous acid water concentration in electrolysis bath 100 becomes lower than the target hypochlorous acid water concentration. Note that, in a case where the water level in electrolysis bath 100 decreases, tap water may be supplied until the water level in electrolysis bath 100 becomes the state of full water.

When the hypochlorous acid water concentration in electrolysis bath 100 becomes lower than the target hypochlorous acid water concentration, controller 600 executes energization to electrode unit 140 to set the hypochlorous acid water concentration in electrolysis bath 100 to the target hypochlorous acid water concentration. A method of calculating the energization time will be described later. As a result, space purification device 2000 can continue releasing a gas containing the hypochlorous acid water.

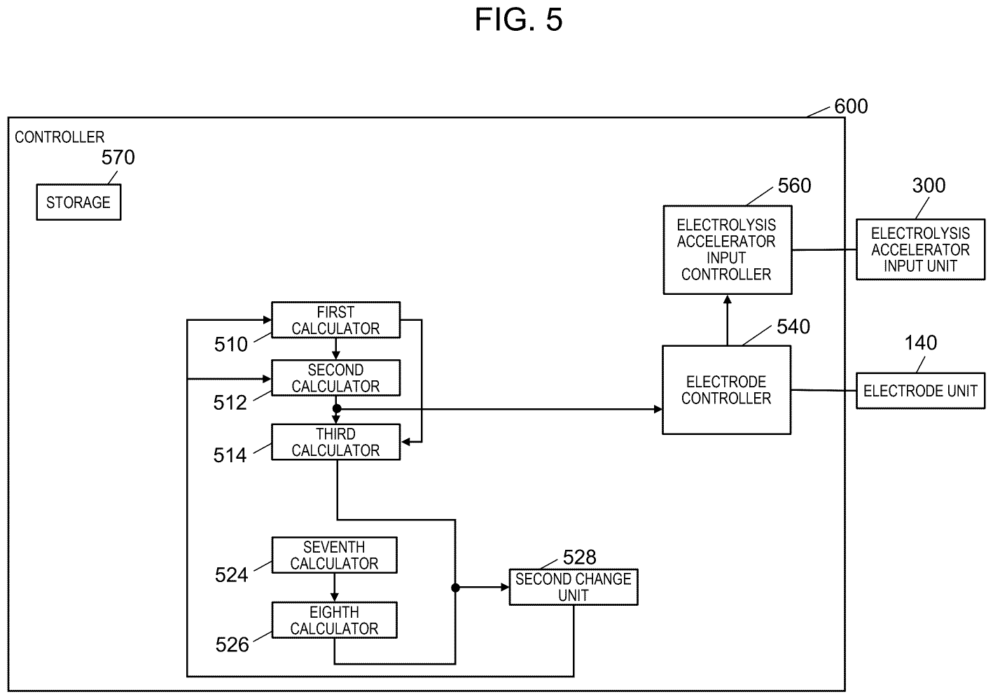

Next, each function and a control flow of controller 600 according to the second exemplary embodiment will be described with reference to FIG. 5. FIG. 5 is a schematic functional block diagram of controller 600 according to the present exemplary embodiment.

In controller 600, Electrolytic water supply controller 550, fourth calculator 516, fifth calculator 518, sixth calculator 520, and first change unit 522 are eliminated from controller 500, and seventh calculator 524, eighth calculator 526, and second change unit 528 are added. That is, controller 600 includes electrolysis accelerator input controller 560, first calculator 510, storage 570, second calculator 512, electrode controller 540, third calculator 514, seventh calculator 524, eighth calculator 526, and second change unit 528.

Electrolysis accelerator input controller 560 controls an input of electrolysis accelerator 310 by electrolysis accelerator input unit 300.

First calculator 510 calculates an increased concentration of hypochlorous acid water per unit time based on a first sodium chloride concentration that is a sodium chloride concentration in electrolysis bath 100 after the sodium chloride concentration is changed due to an input of electrolysis accelerator 310 into electrolysis bath 100, and hypochlorous acid generation efficiency per unit time set in advance. Hereinafter, a specific description will be given using numerical values, but the numerical values are merely examples, and other numerical values may be used.

Firstly, first calculator 510 calculates the first sodium chloride concentration that is a sodium chloride concentration in electrolysis bath 100 after the sodium chloride concentration changes due to the input of electrolysis accelerator 310 into electrolysis bath 100. The first sodium chloride concentration when water is supplied from water supply unit 110 to electrolysis bath 100 from a state where there is no water in electrolysis bath 100 to be a state of full water is 0 [mg/L]. Assuming that a capacity of electrolysis bath 100 is 1 [L] and a weight of electrolysis accelerator 310 is 500 [mg], the calculation formula is as follows: first sodium chloride concentration after sodium chloride concentration changes due to input of electrolysis accelerator 310 into electrolysis bath 100=weight of electrolysis accelerator 310/capacity of electrolysis bath 100=500 [mg/L]/1 [L]=500 [mg/L]. The capacity of electrolysis bath 100 and the weight of electrolysis accelerator 310 are stored in advance in storage 570. Storage 570 is a so-called memory, and stores various values.

Next, first calculator 510 acquires hypochlorous acid generation efficiency per unit time set in advance from storage 570. The hypochlorous acid generation efficiency per unit time is a value determined in advance by an experiment or the like, and can be arbitrarily set. In the present exemplary embodiment, as an example, the hypochlorous acid generation efficiency per unit time is set to 1 [%/min].

Next, first calculator 510 calculates an increased concentration of hypochlorous acid water per unit time. The calculation formula is as follows: increased concentration of hypochlorous acid water per unit time=first sodium chloride concentration×hypochlorous acid generation efficiency per unit time=500 [mg/L]×1 [%/min]=5 [mg/(L×min)].

Second calculator 512 calculates a required energization time for achieving a target hypochlorous acid water concentration based on the target hypochlorous acid water concentration in electrolysis bath 100, a first hypochlorous acid water concentration that is a hypochlorous acid water concentration in electrolysis bath 100 before energization by electrode unit 140, and the increased concentration of hypochlorous acid water per unit time calculated by first calculator 510.

Specifically, first of all, second calculator 512 acquires the target hypochlorous acid water concentration in electrolysis bath 100 from storage 570. In the present exemplary embodiment, the target hypochlorous acid water concentration is 10 [ppm].

Next, second calculator 512 determines the first hypochlorous acid water concentration that is a hypochlorous acid water concentration in electrolysis bath 100 before energization by electrode unit 140. At the first time, since electrolysis bath 100 is in the state of full water and hypochlorous acid does not exist in the water in electrolysis bath 100, second calculator 512 determines 0 [ppm] as the first hypochlorous acid water concentration.

Next, second calculator 512 calculates a required energization time for achieving the target hypochlorous acid water concentration. The calculation formula is as follows: required energization time=(target hypochlorous acid water concentration−hypochlorous acid water concentration before energization)/increased concentration of hypochlorous acid water per unit time calculated by first calculator 510=(10 [ppm]−0 [ppm])/5 [mg/(L×min)]=2 [min].

Electrode controller 540 performs energization in electrode unit 140 for the required energization time calculated by second calculator 512. Specifically, electrode controller 540 performs energization for 2 [min] that is the required energization time calculated by second calculator 512. Thus, the hypochlorous acid water having the target hypochlorous acid water concentration can be generated in electrolysis bath 100.

Third calculator 514 calculates a second sodium chloride concentration that is a sodium chloride concentration in electrolysis bath 100 after energization by electrode unit 140 based on the first sodium chloride concentration, the increased concentration of hypochlorous acid water per unit time, and the required energization time. The sodium chloride concentration in electrolysis bath 100 after energization is lower than the sodium chloride concentration in electrolysis bath 100 before energization.

Specifically, first of all, third calculator 514 acquires the first sodium chloride concentration, the increased concentration of hypochlorous acid water per unit time, and the required energization time. In the present exemplary embodiment, the first sodium chloride concentration=500 [mg/L], the increased concentration of hypochlorous acid water per unit time=5 [mg/(L×min)], and the required energization time=2 [min] are acquired.

Next, third calculator 514 calculates the second sodium chloride concentration. The calculation formula is as follows: second sodium chloride concentration=first sodium chloride concentration−(increased concentration of hypochlorous acid water per unit time×required energization time)=500 [mg/L]−(5 [mg/(L×min)]×2 [min])=490 [mg/L]. As a result, the second sodium chloride concentration that is the sodium chloride concentration in electrolysis bath 100 after energization by electrode unit 140 can be grasped.

Seventh calculator 524 calculates a hypochlorous acid decrease amount decreased from electrolysis bath 100 due to purification by purification unit 401. In the present exemplary embodiment, seventh calculator 524 uses a hypochlorous acid consumption amount table to calculate the hypochlorous acid decrease amount.

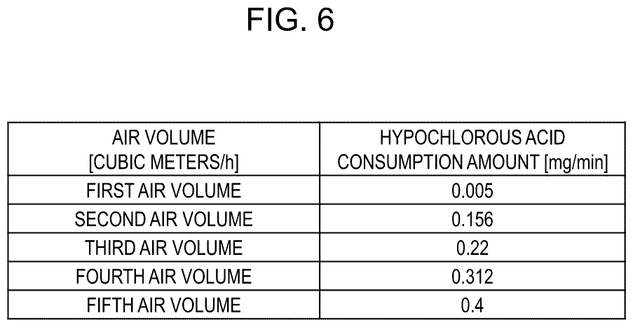

The hypochlorous acid consumption amount table will be described with reference to FIG. 6. FIG. 6 is a diagram illustrating a data structure of a hypochlorous acid consumption amount table stored in storage 570 of controller 600, and is an example of the hypochlorous acid consumption amount table. The hypochlorous acid consumption amount table stores a plurality of hypochlorous acid consumption amounts per predetermined unit time corresponding to predetermined air volumes. The hypochlorous acid consumption amounts per predetermined unit time corresponding to the predetermined air volumes are values determined in advance by an experiment or the like, and can be arbitrarily set.

In the present exemplary embodiment, as an example, when the air volume is a first air volume [cubic meters/h], the hypochlorous acid consumption amount per unit time is 0.005 [mg/min]. Furthermore, when the air volume is a second air volume [cubic meters/h], the hypochlorous acid consumption amount per unit time is 0.156 [mg/min]. When the air volume is a third air volume [cubic meters/h], the hypochlorous acid consumption amount per unit time is 0.22 [mg/min]. When the air volume is a fourth air volume [cubic meters/h], the hypochlorous acid consumption amount per unit time is 0.312 [mg/min]. When the air volume is a fifth air volume [cubic meters/h], the hypochlorous acid consumption amount per unit time is 0.4 [mg/min]. In the hypochlorous acid consumption amount table, as the air volume is larger, the hypochlorous acid consumption amount per corresponding unit time is larger.

In the present exemplary embodiment, the air volume of the fan of purification unit 401 can be set by the user, and the air volume can be set by the user in an air volume setting unit (not illustrated) of space purification device 2000. The air volume that can be set by the user is any one of the first air volume, the second air volume, the third air volume, the fourth air volume, and the fifth air volume. In the present exemplary embodiment, it is assumed that the third air volume is set by the user in the air volume setting unit. Controller 600 acquires the third air volume set by the user, and stores the third air volume in storage 570.

Seventh calculator 524 acquires the hypochlorous acid consumption amount per unit time based on the air volume set by the user and the hypochlorous acid consumption amount table stored in storage 570. Specifically, in a case where the air volume set by the user stored in storage 570 is the third air volume, 0.22 [mg/min] is acquired as the hypochlorous acid consumption amount per unit time from the hypochlorous acid consumption amount table. Seventh calculator 524 calculates a hypochlorous acid decrease amount decreased from electrolysis bath 100 in a predetermined purification time. In the present exemplary embodiment, the predetermined purification time is set to 30 [min]. The calculation formula of the hypochlorous acid decrease amount decreased from electrolysis bath 100 in the predetermined purification time is as follows: hypochlorous acid decrease amount decreased from electrolysis bath 100 in the predetermined purification time=acquired hypochlorous acid consumption amount per unit time×purification time=0.22 [mg/min]×30 [min]=6.6 [mg]. As a result, the hypochlorous acid decrease amount decreased from electrolysis bath 100 in the predetermined purification time can be grasped.

Based on the target hypochlorous acid water concentration and the hypochlorous acid decrease amount calculated by seventh calculator 524, eighth calculator 526 calculates a third hypochlorous acid water concentration that is a hypochlorous acid water concentration in electrolysis bath 100 after the hypochlorous acid is decreased by the purification. The hypochlorous acid water concentration in electrolysis bath 100 after the decrease of the hypochlorous acid by the purification is lower than the hypochlorous acid water concentration (target hypochlorous acid water concentration) in electrolysis bath 100 before the decrease of the hypochlorous acid due to the water supplied by water supply unit 110.

Specifically, first of all, eighth calculator 526 acquires the target hypochlorous acid water concentration and the hypochlorous acid decrease amount calculated by seventh calculator 524. In the present exemplary embodiment, eighth calculator 526 acquires the target hypochlorous acid water concentration=10 [ppm] and the hypochlorous acid decrease amount=6.6 [mg] calculated by seventh calculator 524.

Next, eighth calculator 526 calculates the hypochlorous acid amount in electrolysis bath 100 before the hypochlorous acid decrease amount calculated by seventh calculator 524 decreases. The calculation formula is as follows: hypochlorous acid amount in electrolysis bath 100 before hypochlorous acid decrease amount decreases=target hypochlorous acid water concentration×capacity of electrolysis bath 100=10 [ppm=mg/L]×1 [L]=10 [mg].

Next, eighth calculator 526 calculates the hypochlorous acid amount in electrolysis bath 100 after the hypochlorous acid decrease amount decreases. The calculation formula is as follows: hypochlorous acid amount in electrolysis bath 100 after hypochlorous acid decrease amount decreases=hypochlorous acid amount in electrolysis bath 100 before hypochlorous acid decrease amount decreases−hypochlorous acid decrease amount=10 [mg]−6.6 [mg]=3.4 [mg].

Next, eighth calculator 526 calculates the third hypochlorous acid water concentration. The calculation formula is as follows: third hypochlorous acid water concentration=hypochlorous acid amount in electrolysis bath 100 after the hypochlorous acid decrease amount decreases/capacity of electrolysis bath 100=3.4 [mg]/1 [L]=3.4 [mg/L=ppm]. As a result, it is possible to grasp the hypochlorous acid water concentration in electrolysis bath 100 after the decrease of the hypochlorous acid by the purification.

Second change unit 528 changes the first sodium chloride concentration used for the calculation by first calculator 510 to the second sodium chloride concentration calculated by third calculator 514. Furthermore, second change unit 528 changes the first hypochlorous acid water concentration used for the calculation by second calculator 512 to the third hypochlorous acid water concentration calculated by eighth calculator 526.

Specifically, first of all, second change unit 528 acquires the first sodium chloride concentration before the change and the first hypochlorous acid water concentration before the change. The first sodium chloride concentration before the change and the first hypochlorous acid water concentration before the change are stored in storage 570 by, for example, first calculator 510 or second calculator 512. In the present exemplary embodiment, second change unit 528 acquires the first sodium chloride concentration before the change=500 [mg/L] and the first hypochlorous acid water concentration before the change=0 [ppm].

Second change unit 528 changes the first sodium chloride concentration before the change=500 [mg/L] to the second sodium chloride concentration calculated by third calculator 514=490 [mg/L]. Furthermore, second change unit 528 changes the first hypochlorous acid water concentration before the change=0 [ppm] to the third hypochlorous acid water concentration calculated by eighth calculator 526=3.4 [ppm]. That is, second change unit 528 updates the first sodium chloride concentration to the latest sodium chloride concentration in electrolysis bath 100, and updates the first hypochlorous acid water concentration to the latest hypochlorous acid water concentration in electrolysis bath 100.

First calculator 510 calculates a new increased concentration of hypochlorous acid water per unit time based on a new first sodium chloride concentration (first sodium chloride concentration after the change=the second sodium chloride concentration) changed by second change unit 528 and the hypochlorous acid generation efficiency per unit time set in advance.

Specifically, first of all, first calculator 510 acquires the first sodium chloride concentration after the change and the hypochlorous acid generation efficiency per unit time set in advance. In the present exemplary embodiment, first calculator 510 acquires the first sodium chloride concentration after the change=490 [mg/L] and the hypochlorous acid generation efficiency per unit time set in advance=1 [%/min].

Next, first calculator 510 calculates a new increased concentration of hypochlorous acid water per unit time. A specific calculation formula is as follows: new increased concentration of hypochlorous acid water per unit time=first sodium chloride concentration after the change×hypochlorous acid generation efficiency per unit time=490 [mg/L]×1 [%/min]=4.9 [mg/(L×min)].

Second calculator 512 calculates a new required energization time by electrode unit 140 based on the target hypochlorous acid water concentration in electrolysis bath 100, the new first hypochlorous acid water concentration (the first hypochlorous acid water concentration after the change=the third hypochlorous acid water concentration) changed by second change unit 528, and the new increased concentration of hypochlorous acid water per unit time calculated by first calculator 510.

Specifically, first of all, second calculator 512 acquires the target hypochlorous acid water concentration in electrolysis bath 100, the first hypochlorous acid water concentration after the change, and the new increased concentration of hypochlorous acid water per unit time calculated by first calculator 510. In the present exemplary embodiment, second calculator 512 acquires the target hypochlorous acid water concentration=10 [ppm] in electrolysis bath 100, the first hypochlorous acid water concentration after the change=3.4 [ppm], and the new increased concentration of hypochlorous acid water per unit time=4.9 [mg/(L×min)] calculated by first calculator 510.

Next, second calculator 512 calculates the new required energization time. A specific calculation formula is as follows: new required energization time=(target hypochlorous acid water concentration−first hypochlorous acid water concentration after the change)/new increased concentration of hypochlorous acid water per unit time calculated by first calculator 510=(10 [ppm]−3.4 [ppm])/4.9 [mg/(L×min)]=1.35 [min].

Electrode controller 540 performs energization in electrode unit 140 for the new required energization time calculated by second calculator 512. In the present exemplary embodiment, second calculator 512 performs energization in electrode unit 140 for the new required energization time=1.35 [min]. Thus, the hypochlorous acid water having the target hypochlorous acid water concentration can be generated again in electrolysis bath 100.

Thereafter, controller 500 repeats the calculation by third calculator 514, the calculation by seventh calculator 524, the calculation by eighth calculator 526, the change by second change unit 528, the calculation by first calculator 510, and the calculation by second calculator 512. That is, controller 600 repeats the change of the first sodium chloride concentration and the change of the first hypochlorous acid water concentration by second change unit 528, the calculation of the new increased concentration of hypochlorous acid water per unit time by first calculator 510, the calculation of the new required energization time by second calculator 512, and the control of energizing for the new required energization time by electrode controller 540. As a result, the hypochlorous acid water concentration in electrolysis bath 100 can be continuously set to the target hypochlorous acid water concentration.

Each functional block of controller 600 can also be implemented in various forms by a combination of hardware and software.

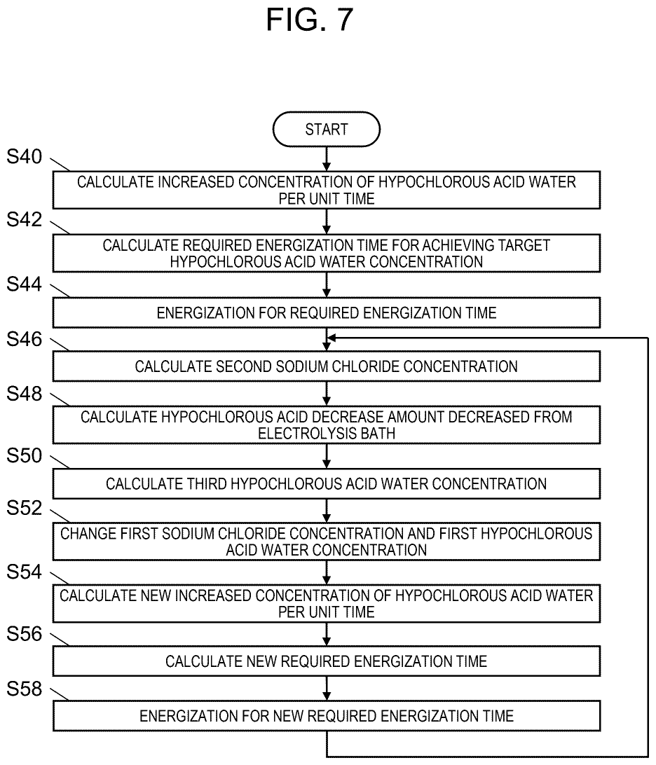

A flowchart of controller 600 having the above configuration will be described. FIG. 7 is a flowchart illustrating control of controller 600 according to the present exemplary embodiment.

Firstly, first calculator 510 calculates an increased concentration of hypochlorous acid water per unit time (S40). Second calculator 512 calculates a required energization time for achieving the target hypochlorous acid water concentration (S42). Electrode controller 540 performs energization in electrode unit 140 for the required energization time calculated by second calculator 512 (S44). Thus, the hypochlorous acid water having the target hypochlorous acid water concentration can be generated in electrolysis bath 100.

Third calculator 514 calculates a second sodium chloride concentration that is a sodium chloride concentration in electrolysis bath 100 after energization by electrode unit 140 (S46). As a result, the sodium chloride concentration in electrolysis bath 100 after energization by electrode unit 140 can be grasped.

Seventh calculator 524 calculates a hypochlorous acid decrease amount decreased from electrolysis bath 100 due to purification by purification unit 401 (S48). As a result, the hypochlorous acid decrease amount decreased from electrolysis bath 100 can be grasped.

Eighth calculator 526 calculates a third hypochlorous acid water concentration that is a hypochlorous acid water concentration in electrolysis bath 100 after decrease of the hypochlorous acid by the purification (S50). As a result, it is possible to grasp the hypochlorous acid water concentration in electrolysis bath 100 after the decrease of the hypochlorous acid by the purification.

Second change unit 528 changes the first sodium chloride concentration to the second sodium chloride concentration calculated by third calculator 514, and changes the first hypochlorous acid water concentration to the third hypochlorous acid water concentration calculated by eighth calculator 526 (S52).

First calculator 510 calculates a new increased concentration of hypochlorous acid water per unit time based on a new first sodium chloride concentration changed by second change unit 528 and hypochlorous acid generation efficiency per unit time set in advance (S54).

Second calculator 512 calculates a new required energization time by electrode unit 140 based on the target hypochlorous acid water concentration in electrolysis bath 100, a new first hypochlorous acid water concentration changed by second change unit 528, and the new increased concentration of hypochlorous acid water per unit time (S56).

Electrode controller 540 performs energization in electrode unit 140 for the new required energization time calculated by second calculator 512 (S58). Thus, the hypochlorous acid water having the target hypochlorous acid water concentration can be generated again in electrolysis bath 100. Thereafter, the process returns to step S46.

Controller 600 repeats steps S46 to S58. As a result, the hypochlorous acid water concentration in electrolysis bath 100 can be continuously set to the target hypochlorous acid water concentration.

As described above, even in space purification device 2000 not including purification bath 200, the hypochlorous acid water concentration of electrolysis bath 100 can be continuously set to the target hypochlorous acid water concentration.

### Third Exemplary Embodiment

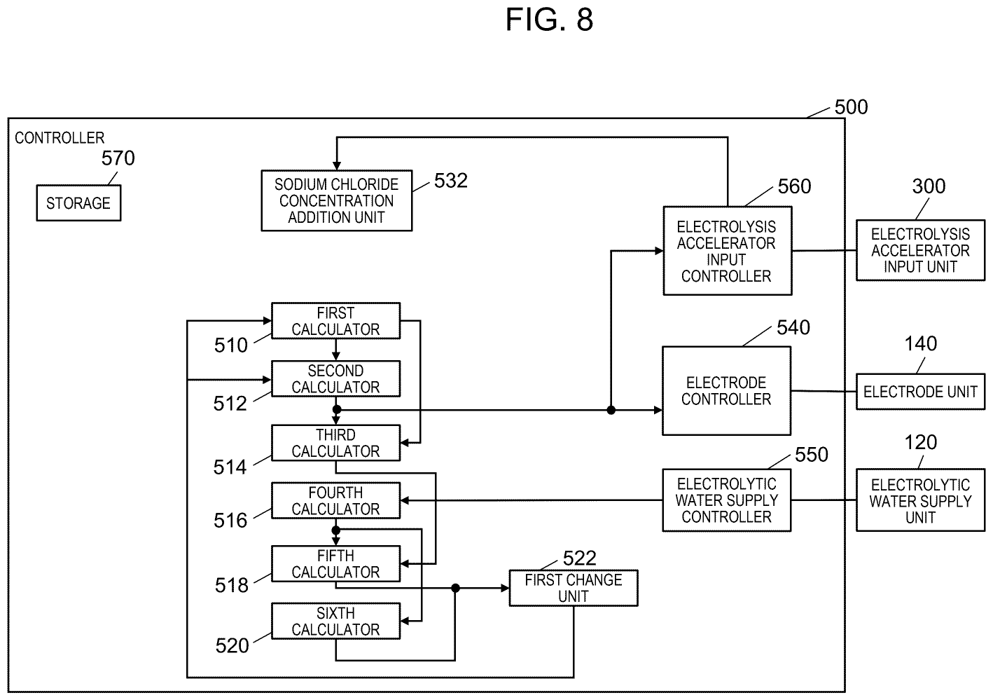

A third exemplary embodiment relates to input control of electrolysis accelerator 310. FIG. 8 is a schematic functional block diagram of controller 500 according to the third exemplary embodiment. The internal configuration of a space purification device in the third exemplary embodiment is the same as that of space purification device 1000 in the first exemplary embodiment. Furthermore, a schematic functional block diagram of controller 500 in the third exemplary embodiment is partially different from that in the first exemplary embodiment. Specifically, the control contents of electrolysis accelerator input controller 560 are added, and sodium chloride concentration addition unit 532 is added.

When a required energization time calculated by second calculator 512 is longer than a longest energization time, electrolysis accelerator input controller 560 gives an input instruction to electrolysis accelerator input unit 300, and electrolysis accelerator input unit 300 inputs electrolysis accelerator 310. That is, when the required energization time calculated by second calculator 512 is longer than the longest energization time, electrolysis accelerator input unit 300 inputs electrolysis accelerator 310.

The longest energization time is for determining whether or not it is necessary to input electrolysis accelerator 310 into electrolysis bath 100 due to a decrease in sodium chloride in electrolysis bath 100, and can be arbitrarily set. In the present exemplary embodiment, for example, when the longest energization time=10 [min], if the required energization time is longer than 10 [min], then electrolysis accelerator input unit 300 inputs electrolysis accelerator 310.

By performing energization in electrode unit 140, water containing chloride ions is electrolyzed to generate hypochlorous acid. In the generation of the hypochlorous acid, sodium chloride is consumed, and a sodium chloride concentration in electrolysis bath 100 decreases. That is, by continuing the generation of the hypochlorous acid, the sodium chloride concentration in electrolysis bath 100 continues to decrease.

As the sodium chloride concentration in electrolysis bath 100 decreases, the increased concentration of hypochlorous acid water per unit time decreases. As a result, the required energization time increases, and the hypochlorous acid water having the target hypochlorous acid water concentration cannot be stably supplied to purification bath 200. Therefore, when the required energization time calculated by second calculator 512 is longer than the longest energization time, electrolysis accelerator input unit 300 inputs electrolysis accelerator 310. As a result, the sodium chloride concentration in electrolysis bath 100 increases, and the required energization time can be shortened. As a result, the hypochlorous acid water having the target hypochlorous acid water concentration can be stably supplied to purification bath 200.

The addition of the sodium chloride concentration in electrolysis bath 100 in a case where electrolysis accelerator 310 is input by electrolysis accelerator input unit 300 is performed by sodium chloride concentration addition unit 532.

Sodium chloride concentration addition unit 532 adds an increase theoretical value of the sodium chloride concentration due to an input of electrolysis accelerator 310 to the sodium chloride concentration in electrolysis bath 100 after the input of electrolysis accelerator 310 by electrolysis accelerator input unit 300. Hereinafter, a description will be given using numerical values as specific examples, but the numerical values are merely examples.

In the present exemplary embodiment, it is assumed that one tablet of electrolysis accelerator 310 is input by electrolysis accelerator input unit 300, and the weight (weight of sodium chloride) of one tablet of electrolysis accelerator 310 is 500 [mg]. The weight of electrolysis accelerator 310 input by the electrolysis accelerator input unit 300 is stored in advance in storage 570. Furthermore, as described above, the capacity=1 [L] of electrolysis bath 100 is also stored in advance in storage 570.

Firstly, sodium chloride concentration addition unit 532 calculates the increase theoretical value of the sodium chloride concentration due to the input of electrolysis accelerator 310. The weight of electrolysis accelerator 310 input by electrolysis accelerator input unit 300 is 500 [mg], the capacity of electrolysis bath 100 is 1 [L], and the calculation formula is as follows: increase theoretical value=weight of electrolysis accelerator 310 input by electrolysis accelerator input unit 300/capacity of electrolysis bath 100=500 [mg]/1 [L]=500 [mg/L].

Next, sodium chloride concentration addition unit 532 adds the increase theoretical value of the sodium chloride concentration due to the input of electrolysis accelerator 310 to the sodium chloride concentration in electrolysis bath 100. As a result, it is possible to grasp the sodium chloride concentration in electrolysis bath 100 after the input of electrolysis accelerator 310.

An example of a control flow will be described below. For example, the first sodium chloride concentration is 100 [mg/L], the hypochlorous acid generation efficiency per unit time is 1 [%/min], the target hypochlorous acid water concentration is 20 [ppm], and the first hypochlorous acid water concentration is 8 [ppm].

Firstly, first calculator 510 calculates an increased concentration of hypochlorous acid water per unit time. The calculation formula is as follows: increased concentration of hypochlorous acid water per unit time=first sodium chloride concentration×hypochlorous acid generation efficiency per unit time=100 [mg/L]×1 [%/min]=1 [mg/(L×min)].

Second calculator 512 calculates a required energization time for achieving the target hypochlorous acid water concentration. The calculation formula is as follows: required energization time=(target hypochlorous acid water concentration−first hypochlorous acid water concentration)/increased concentration of hypochlorous acid water per unit time=(20 [ppm]−8 [ppm])/1 [mg/(L×min)]=12 [min].

Since the required energization time=12 [min] calculated by second calculator 512 is longer than the longest energization time=10 [min], electrolysis accelerator input controller 560 gives an input instruction to electrolysis accelerator input unit 300, and electrolysis accelerator input unit 300 inputs electrolysis accelerator 310.

Next, sodium chloride concentration addition unit 532 adds the increase theoretical value of the sodium chloride concentration due to the input of electrolysis accelerator 310 to the first sodium chloride concentration. The calculation formula is as follows: first sodium chloride concentration after addition=first sodium chloride concentration before addition+increase theoretical value=100 [mg/L]+500 [mg/L]=600 [mg/L]. That is, the first sodium chloride concentration is updated to 600 [mg/L].

First calculator 510 calculates a new increased concentration of hypochlorous acid water per unit time based on a new first sodium chloride concentration and hypochlorous acid generation efficiency per unit time set in advance. The calculation formula is as follows: new increased concentration of hypochlorous acid water per unit time=first sodium chloride concentration after addition by sodium chloride concentration addition unit 532×hypochlorous acid generation efficiency per unit time=600 [mg/L]×1 [%/min]=6 [mg/(L×min)].

Second calculator 512 calculates a new required energization time based on the target hypochlorous acid water concentration in electrolysis bath 100, the first hypochlorous acid water concentration, and the new increased concentration of hypochlorous acid water per unit time calculated by first calculator 510. The calculation formula is as follows: new required energization time=(target hypochlorous acid water concentration−first hypochlorous acid water concentration)/new increased concentration of hypochlorous acid water per unit time=(20 [ppm]−8 [ppm])/6 [mg/(L×min)]=2 [min].

Since the new required energization time=2 [min] calculated by second calculator 512 is less than or equal to the longest energization time=10 [min], electrolysis accelerator input controller 560 does not give an input instruction to electrolysis accelerator input unit 300, and electrolysis accelerator input unit 300 does not input electrolysis accelerator 310.

Electrode controller 540 performs energization in electrode unit 140 for the new required energization time calculated by second calculator 512. As a result, the hypochlorous acid water having the target hypochlorous acid water concentration can be generated in electrolysis bath 100 in less than or equal to the longest energization time. As a result, the hypochlorous acid water having the target hypochlorous acid water concentration can be stably supplied to purification bath 200.

### Fourth Exemplary Embodiment

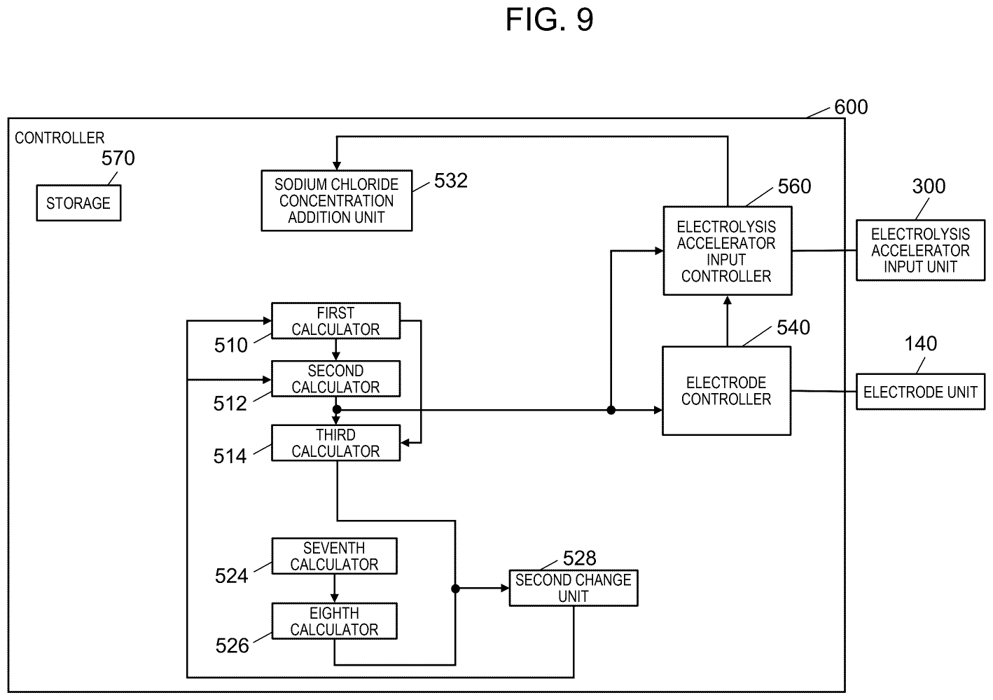

A fourth exemplary embodiment also relates to input control of electrolysis accelerator 310. FIG. 9 is a schematic functional block diagram of controller 600 according to the fourth exemplary embodiment. The internal configuration of a space purification device in the fourth exemplary embodiment is the same as that of space purification device 2000 in the second exemplary embodiment. Furthermore, a schematic functional block diagram of controller 600 in the fourth exemplary embodiment is partially different from that in the second exemplary embodiment. Specifically, the control contents of electrolysis accelerator input controller 560 are added, and sodium chloride concentration addition unit 532 is added. Since the additional contents of the control of electrolysis accelerator input controller 560 and the additional contents of sodium chloride concentration addition unit 532 are the same as those in the third exemplary embodiment, the description thereof will be omitted.

Also in space purification device 2000, by performing energization in electrode unit 140, water containing chloride ions is electrolyzed to generate hypochlorous acid. In the generation of the hypochlorous acid, sodium chloride is consumed, and a sodium chloride concentration in electrolysis bath 100 decreases. That is, by continuing the generation of the hypochlorous acid, the sodium chloride concentration in electrolysis bath 100 continues to decrease.

As the sodium chloride concentration in electrolysis bath 100 decreases, the increased concentration of hypochlorous acid water per unit time decreases. As a result, the required energization time increases, and the hypochlorous acid water concentration in electrolysis bath 100 cannot be stably set to the target hypochlorous acid water concentration. Therefore, when the required energization time calculated by second calculator 512 is longer than the longest energization time, electrolysis accelerator input unit 300 inputs electrolysis accelerator 310. As a result, the sodium chloride concentration in electrolysis bath 100 increases, the required energization time can be shortened, and as a result, the hypochlorous acid water concentration in electrolysis bath 100 can be stably set to the target hypochlorous acid water concentration.

### Fifth Exemplary Embodiment

A fifth exemplary embodiment relates to control of a sodium chloride concentration addition unit. The control contents of sodium chloride concentration addition unit 532 of the fifth exemplary embodiment are different from those of the third exemplary embodiment or the fourth exemplary embodiment. Details will be described below.

After the input of electrolysis accelerator 310 by electrolysis accelerator input unit 300, sodium chloride concentration addition unit 532 performs, a predetermined number of times, processing of adding a divided increase value to a sodium chloride concentration in electrolysis bath 100 every unit time, and the divided increase value is obtained by dividing the increase theoretical value of the sodium chloride concentration due to the input of electrolysis accelerator 310 by the predetermined number of times. This will be specifically described below.

The predetermined number of times is a value determined in advance by an experiment or the like, and can be arbitrarily set. The predetermined number of times is stored in storage 570.

Furthermore, storage 570 also stores a time (Hereinafter, it is described as increase completion time) until the sodium chloride concentration in electrolysis bath 100 is increased by the increase theoretical value. The increase completion time is a time from when electrolysis accelerator 310 is input into electrolysis bath 100 to when electrolysis accelerator 310 is completely dissolved in electrolysis bath 100, and is a value determined in advance by an experiment or the like, and can be arbitrarily set.

When electrolysis accelerator 310 is input at a sodium chloride concentration in electrolysis bath 100 before the input of electrolysis accelerator 310, electrolysis accelerator 310 starts to dissolve, and the sodium chloride concentration in electrolysis bath 100 increases with the lapse of time. Then, when the increase completion time elapses, electrolysis accelerator 310 finishes dissolving, and the sodium chloride concentration in electrolysis bath 100 after the completion of dissolution of electrolysis accelerator 310 is increased from a sodium chloride concentration in electrolysis bath 100 before the input of electrolysis accelerator 310 by the increase theoretical value. In the present exemplary embodiment, as an example, the increase theoretical value=500 [mg/L], the predetermined number of times=4, and the increase completion time=2 [min] are set.

First, sodium chloride concentration addition unit 532 calculates a unit time. The calculation formula is as follows: unit time=increase completion time/predetermined number of times=2 [min]/4=0.5 [min].

Sodium chloride concentration addition unit 532 calculates the divided increase value. The calculation formula is: divided increase value=increase theoretical value/predetermined number of times=500 [mg/L]/4=125 [mg/L].

Sodium chloride concentration addition unit 532 performs division processing of adding the divided increase value to the sodium chloride concentration in electrolysis bath 100 after a lapse of the unit time from the input of electrolysis accelerator 310. That is, sodium chloride concentration addition unit 532 performs the division processing of adding 125 [mg/L] to the sodium chloride concentration in electrolysis bath 100 after 0.5 [min] elapses from the input of electrolysis accelerator 310.

Sodium chloride concentration addition unit 532 performs the division processing every unit time elapses, and the division processing is performed for the predetermined number of times. That is, sodium chloride concentration addition unit 532 performs the division processing for four times. As a result, the sodium chloride concentration in electrolysis bath 100 after the elapse of the increase completion time is a concentration increased by the increase theoretical value from the sodium chloride concentration in electrolysis bath 100 before the input of electrolysis accelerator 310.

In the third exemplary embodiment or the fourth exemplary embodiment, in order to achieve simple control, the increase theoretical value of the sodium chloride concentration is added to the sodium chloride concentration in electrolysis bath 100 immediately after the input of electrolysis accelerator 310, but the present exemplary embodiment has control contents more suitable for the actual movement of the sodium chloride concentration. That is, since the sodium chloride concentration in electrolysis bath 100 can be grasped with high accuracy, the hypochlorous acid water having the target hypochlorous acid water concentration can be generated with high accuracy.

### Sixth Exemplary Embodiment

A sixth exemplary embodiment relates to input control of electrolysis accelerator 310. The internal configuration of a space purification device in the sixth exemplary embodiment is the same as that of space purification device 1000 in the third exemplary embodiment, and the schematic functional block diagram of controller 500 in the sixth exemplary embodiment is also the same as that in the third exemplary embodiment. The input control of the electrolysis accelerator in the sixth exemplary embodiment is partially different from the input control of electrolysis accelerator 310 in the third exemplary embodiment. Hereinafter, differences from the input control of electrolysis accelerator 310 in the third exemplary embodiment will be mainly described, and the description of the same control contents will be omitted.

Electrolytic water supply controller 550 supplies hypochlorous acid water to purification bath 200 by electrolytic water supply unit 120 every first time.

The first time is a value determined in advance by an experiment or the like, and can be arbitrarily set. The first time is stored in storage 570. Since the hypochlorous acid water is supplied to purification bath 200 by electrolytic water supply unit 120 every first time, the space can be continuously purified using the hypochlorous acid water stored in purification bath 200. The first time is a time during which the hypochlorous acid water is reliably stored in purification bath 200 even after the first time has elapsed from the previous supply of the hypochlorous acid water, and is stored in storage 570.

When the required energization time calculated by second calculator 512 is longer than the first time, electrolysis accelerator input controller 560 gives an input instruction to electrolysis accelerator input unit 300, and electrolysis accelerator input unit 300 inputs electrolysis accelerator 310. That is, when the required energization time calculated by second calculator 512 is longer than the first time, electrolysis accelerator input unit 300 inputs electrolysis accelerator 310.

When the required energization time is longer than the first time, the hypochlorous acid water concentration of electrolysis bath 100 when the hypochlorous acid water is supplied to purification bath 200 by electrolytic water supply unit 120 becomes lower than the target hypochlorous acid concentration. That is, the hypochlorous acid water concentration in purification bath 200 becomes lower than the target hypochlorous acid concentration, and an amount of hypochlorous acid released to the outside decreases. When the hypochlorous acid water concentration in purification bath 200 becomes lower than the target hypochlorous acid concentration, there is a possibility that the inside of the space cannot be efficiently purified.

As described above, by continuing the generation of hypochlorous acid, the sodium chloride concentration in electrolysis bath 100 continues to decrease. As the sodium chloride concentration in electrolysis bath 100 decreases, the increased concentration of hypochlorous acid water per unit time decreases, and as a result, the required energization time becomes longer, and when the required energization time becomes longer than the first time, the hypochlorous acid water having the target hypochlorous acid water concentration cannot be supplied to purification bath 200. Therefore, when the required energization time calculated by second calculator 512 is longer than the first time, electrolysis accelerator input unit 300 inputs electrolysis accelerator 310. As a result, the sodium chloride concentration in electrolysis bath 100 increases, and the required energization time can be shortened. As a result, the hypochlorous acid water having the target hypochlorous acid water concentration can be supplied to purification bath 200.

An example of a control flow will be described below.

Firstly, first calculator 510 calculates an increased concentration of hypochlorous acid water per unit time. Second calculator 512 calculates a required energization time for achieving the target hypochlorous acid water concentration.

When the required energization time calculated by second calculator 512 is longer than the first time, electrolysis accelerator input controller 560 gives an input instruction to electrolysis accelerator input unit 300, and electrolysis accelerator input unit 300 inputs electrolysis accelerator 310.

Next, sodium chloride concentration addition unit 532 adds the increase theoretical value of the sodium chloride concentration due to the input of electrolysis accelerator 310 to the first sodium chloride concentration. The first sodium chloride concentration is added by sodium chloride concentration addition unit 532 to become a new first sodium chloride concentration. That is, as an addition method by sodium chloride concentration addition unit 532, the addition method described in the third exemplary embodiment or the fifth exemplary embodiment can be used.

First calculator 510 calculates a new increased concentration of hypochlorous acid water per unit time based on a new first sodium chloride concentration and hypochlorous acid generation efficiency per unit time set in advance.

Second calculator 512 calculates a new required energization time based on the target hypochlorous acid water concentration in electrolysis bath 100, the first hypochlorous acid water concentration, and the new increased concentration of hypochlorous acid water per unit time calculated by first calculator 510.

When the new required energization time calculated by second calculator 512 is less than or equal to the first time, electrolysis accelerator input controller 560 does not give an input instruction to electrolysis accelerator input unit 300, and electrolysis accelerator input unit 300 does not input electrolysis accelerator 310.

Electrode controller 540 performs energization in electrode unit 140 for the new required energization time calculated by second calculator 512. As a result, the hypochlorous acid water having the target hypochlorous acid water concentration can be generated in electrolysis bath 100 in the first time or less, and as a result, the hypochlorous acid water having the target hypochlorous acid water concentration can be stably supplied to purification bath 200.

### Seventh Exemplary Embodiment

A seventh exemplary embodiment relates to energization control and input control of electrolysis accelerator 310 by electrode controller 540. The control contents of electrode controller 540 and electrolysis accelerator input controller 560 of the seventh exemplary embodiment are partially different from the control contents of the third exemplary embodiment, the fourth exemplary embodiment, and the sixth exemplary embodiment. Differences will be mainly described.

Electrode controller 540 performs energization control to alternately perform energization and non-energization by electrode unit 140. By providing a non-energization time, the electrode life of electrode unit 140 can be lengthened.

When a timing of the instruction to input the electrolysis accelerator to electrolysis accelerator input unit 300 is in an energization state, electrolysis accelerator input controller 560 waits until the non-energization time, and performs the instruction to input the electrolysis accelerator to electrolysis accelerator input unit 300 in a non-energization state. Then, electrolysis accelerator input unit 300 inputs electrolysis accelerator 310. That is, when a timing of inputting electrolysis accelerator 310 is in the energization state, electrolysis accelerator input unit 300 waits until the non-energization state, and inputs electrolysis accelerator 310 in the non-energization state.

When electrolysis accelerator 310 is input in the energization state, the sodium chloride concentration in electrolysis bath 100 changes in the energization state. That is, the hypochlorous acid concentration in electrolysis bath 100 after completion of energization is not the target hypochlorous acid concentration. By performing this control, it is possible to stably set the hypochlorous acid concentration in electrolysis bath 100 after completion of energization to the target hypochlorous acid concentration while prolonging the life of electrode unit 140.

### Eighth Exemplary Embodiment

An eighth exemplary embodiment also relates to energization control and input control of electrolysis accelerator 310 by electrode controller 540. The control contents of electrode controller 540 and electrolysis accelerator input controller 560 of the eighth exemplary embodiment are also partially different from the control contents of the third exemplary embodiment, the fourth exemplary embodiment, and the sixth exemplary embodiment. Differences will be mainly described.

Electrode controller 540 performs energization control in which energization and non-energization by electrode unit 140 are alternately performed, an energization direction is reversed from a first energization direction after a total energization time in the first energization direction has elapsed a second time, and a first non-energization time is provided after the reverse of the energization direction. Details will be described below.

Electrode controller 540 alternately performs energization and non-energization by electrode unit 140, and can switch the energization direction of electrode unit 140 between the first energization direction and an energization direction opposite to the first energization direction. In the present exemplary embodiment, as an example, the first energization direction is defined as an energization direction from a positive electrode to a negative electrode of electrode unit 140, and the energization direction opposite to the first energization direction is defined as an energization direction from the negative electrode to the positive electrode of electrode unit 140.

Electrode controller 540 reverses the energization direction from the first energization direction after the total energization time in the first energization direction has elapsed the second time. Specifically, when the total energization time in the first energization direction by electrode unit 140 becomes the second time, electrode controller 540 completes the energization while keeping the energization in the first energization direction. After completion of energization in the first energization direction, electrode controller 540 causes electrode unit 140 to provide a non-energization time longer than or equal to at least the first non-energization time. Electrode controller 540 reverses the energization direction of electrode unit 140 from the first energization direction before the start of the next energization, and instructs the next energization.

It is desirable to periodically change the energization direction of electrode unit 140. This is because when the energization is continued without changing the energization direction, a scale adheres to a surface of electrode unit 140, and the efficiency of electrolysis is reduced and the electrode life is reached at an early stage. That is, the second time is a time provided for suppressing a reduction in efficiency of electrolysis and suppressing early reaching of the electrode life, is a value determined in advance by an experiment or the like, and can be arbitrarily set. The second time is stored in storage 570.

Furthermore, when the reverse of the energization direction of electrode unit 140 is performed without providing the non-energization time longer than or equal to the first non-energization time, the charged charge of the electrode generated in electrode unit 140 peels a catalyst layer on the electrode surface. As a result, the electrode deteriorates, and the efficiency of electrolysis reduces and the electrode life reaches an early stage. That is, the first non-energization time is a time provided for suppressing a reduction in efficiency of electrolysis and suppressing early reaching of the electrode life, is a value determined in advance by an experiment or the like, and can be arbitrarily set. The first non-energization time is stored in storage 570.

When a timing of the instruction to input the electrolysis accelerator to electrolysis accelerator input unit 300 is in an energization state, electrolysis accelerator input controller 560 waits until the non-energization time, and performs the instruction to input the electrolysis accelerator to electrolysis accelerator input unit 300 in a non-energization state. Then, electrolysis accelerator input unit 300 inputs electrolysis accelerator 310. That is, electrolysis accelerator input unit 300 inputs electrolysis accelerator 310 in the non-energization state. The reason why it is better to input electrolysis accelerator 310 in the non-energization state is the same as in the seventh exemplary embodiment. That is, by performing this control, it is possible to stably set the hypochlorous acid concentration in electrolysis bath 100 after completion of energization to the target hypochlorous acid concentration while prolonging the life of electrode unit 140.

Although the present disclosure has been described above based on the exemplary embodiments, the present disclosure is not limited to the exemplary embodiments described above, and various modifications can be made without departing from the gist of the present disclosure. Furthermore, the numerical values mentioned in the above exemplary embodiments are merely examples, and it is needless to say that the present disclosure is not limited to adopting the numerical values used in the description of the exemplary embodiments.

For example, in the first exemplary embodiment, the configuration in which electrolytic water supply unit 120 includes water supply pump 122, water supply pipe 124, and supply port 126 has been described, but any configuration other than this configuration may be adopted as long as hypochlorous acid water can be supplied from electrolysis bath 100 to purification bath 200.

### SUMMARY OF THE INVENTION

A space purification device according to the present disclosure includes: an electrolysis bath that mixes an electrolysis accelerator and water; a water supply unit that supplies the water to the electrolysis bath; an electrode unit that generates hypochlorous acid water from the electrolysis accelerator and the water mixed in the electrolysis bath; a first calculator that calculates an increased concentration of hypochlorous acid water per unit time based on (i) a first sodium chloride concentration that is a sodium chloride concentration in the electrolysis bath after the sodium chloride concentration has changed due to an input of the electrolysis accelerator into the electrolysis bath and (ii) hypochlorous acid generation efficiency per unit time set in advance; a second calculator that calculates, based on a target hypochlorous acid water concentration in the electrolysis bath, a first hypochlorous acid water concentration that is a hypochlorous acid water concentration in the electrolysis bath before energization by the electrode unit, and the increased concentration of hypochlorous acid water per unit time calculated by the first calculator, a required energization time for achieving the target hypochlorous acid water concentration; and an electrode controller that performs energization in the electrode unit for the required energization time calculated by the second calculator. Thus, hypochlorous acid water having the target hypochlorous acid water concentration can be generated in the electrolysis bath.

Furthermore, the space purification device may further include a third calculator that calculates a second sodium chloride concentration that is the sodium chloride concentration in the electrolysis bath after the energization by the electrode unit based on the first sodium chloride concentration, the increased concentration of hypochlorous acid water per unit time, and the required energization time. As a result, the second sodium chloride concentration that is a sodium chloride concentration in the electrolysis bath after energization by the electrode unit can be grasped.

Furthermore, the space purification device may further include: a purification bath that stores the hypochlorous acid water generated in the electrolysis bath; an electrolytic water supply unit that supplies the hypochlorous acid water from the electrolysis bath to the purification bath; and a purification unit that purifies a space using the hypochlorous acid water stored in the purification bath. As a result, the space can be purified.

Furthermore, the space purification device may further include: an electrolytic water supply controller that supplies electrolytic water to the purification bath by the electrolytic water supply unit; a fourth calculator that calculates an amount of supplied electrolytic water by the electrolytic water supply unit; and a fifth calculator that calculates a third sodium chloride concentration that is the sodium chloride concentration in the electrolysis bath after a supply of the electrolytic water by the electrolytic water supply unit based on the second sodium chloride concentration calculated by the third calculator, the amount of supplied electrolytic water calculated by the fourth calculator, and a capacity of the electrolysis bath. As a result, it is possible to grasp the third sodium chloride concentration that is a sodium chloride concentration in the electrolysis bath after the supply of the electrolytic water by the electrolytic water supply unit.

Furthermore, the space purification device may further include a sixth calculator that calculates a second hypochlorous acid water concentration that is the hypochlorous acid water concentration in the electrolysis bath after the supply of the electrolytic water by the electrolytic water supply unit based on the target hypochlorous acid water concentration, the amount of supplied electrolytic water, and the capacity of the electrolysis bath. As a result, the second hypochlorous acid water concentration that is a hypochlorous acid water concentration in the electrolysis bath after the supply of hypochlorous acid water can be grasped.

Furthermore, the space purification device may further include a first change unit that changes the first sodium chloride concentration to the third sodium chloride concentration calculated by the fifth calculator and changes the first hypochlorous acid water concentration to the second hypochlorous acid water concentration calculated by the sixth calculator, in which the first calculator may calculate a new increased concentration of hypochlorous acid water per unit time based on (i) a new first sodium chloride concentration changed by the first change unit and (ii) the hypochlorous acid generation efficiency per unit time set in advance, the second calculator may calculate a new required energization time by the electrode unit based on the target hypochlorous acid water concentration in the electrolysis bath, a new first hypochlorous acid water concentration changed by the first change unit, and the new increased concentration of hypochlorous acid water per unit time calculated by the first calculator, and the electrode controller may be configured to perform energization in the electrode unit for the new required energization time calculated by the second calculator. Thus, the hypochlorous acid water having the target hypochlorous acid water concentration can be generated again in the electrolysis bath.

Furthermore, the space purification device may be configured to repeat the change of the first sodium chloride concentration and the change of the first hypochlorous acid water concentration by the first change unit, the calculation of the new increased concentration of hypochlorous acid water per unit time by the first calculator, the calculation of the new required energization time by the second calculator, and the control of energization for the new required energization time by the electrode controller. As a result, the hypochlorous acid water concentration in the electrolysis bath can be continuously set to the target hypochlorous acid water concentration.

Furthermore, the space purification device may further include a purification unit that purifies a space using the hypochlorous acid water in the electrolysis bath. As a result, the space can be purified.

Furthermore, the space purification device may further include: a seventh calculator that calculates a hypochlorous acid decrease amount decreased from the electrolysis bath due to the purification by the purification unit; and an eighth calculator that calculates a third hypochlorous acid water concentration that is the hypochlorous acid water concentration in the electrolysis bath after decrease of hypochlorous acid by the purification based on the target hypochlorous acid water concentration and the hypochlorous acid decrease amount calculated by the seventh calculator. As a result, it is possible to grasp the third hypochlorous acid water concentration that is a hypochlorous acid water concentration in the electrolysis bath after decrease of hypochlorous acid by the purification.

Furthermore, the space purification device may further includes a second change unit that changes the first sodium chloride concentration to the second sodium chloride concentration calculated by the third calculator and changes the first hypochlorous acid water concentration to the third hypochlorous acid water concentration calculated by the eighth calculator, in which the first calculator may calculate a new increased concentration of hypochlorous acid water per unit time based on (i) a new first sodium chloride concentration changed by the second change unit and (ii) the hypochlorous acid generation efficiency per unit time set in advance, the second calculator may calculate a new required energization time by the electrode unit based on the target hypochlorous acid water concentration in the electrolysis bath, a new first hypochlorous acid water concentration changed by the second change unit, and the new increased concentration of hypochlorous acid water per unit time calculated by the first calculator, and the electrode controller may be configured to perform energization in the electrode unit for the new required energization time calculated by the second calculator. Thus, the hypochlorous acid water having the target hypochlorous acid water concentration can be generated again in the electrolysis bath.

Furthermore, the space purification device may be configured to repeat the change of the first sodium chloride concentration and the change of the first hypochlorous acid water concentration by the second change unit, the calculation of the new increased concentration of hypochlorous acid water per unit time by the first calculator, the calculation of the new required energization time by the second calculator, and the control of energization for the new required energization time by the electrode controller. As a result, the hypochlorous acid water concentration in the electrolysis bath can be continuously set to the target hypochlorous acid water concentration.

Furthermore, the space purification device may further include an electrolysis accelerator input unit that inputs the electrolysis accelerator into the electrolysis bath, in which the electrolysis accelerator input unit may be configured to input the electrolysis accelerator when the required energization time calculated by the second calculator is longer than a longest energization time. As a result, the hypochlorous acid water concentration in the electrolysis bath can be stably set to the target hypochlorous acid water concentration.

Furthermore, the space purification device may further include an electrolysis accelerator input unit that inputs the electrolysis accelerator into the electrolysis bath, in which the electrolytic water supply controller may supply electrolytic water to the purification bath by the electrolytic water supply unit every first time, and the electrolysis accelerator input unit may be configured to input the electrolysis accelerator when the required energization time calculated by the second calculator is longer than the first time. As a result, it is possible to stably supply the electrolytic water having the target hypochlorous acid water concentration to the purification bath.

The space purification device may further include a sodium chloride concentration addition unit that adds an increase theoretical value of the sodium chloride concentration due to the input of the electrolysis accelerator to the sodium chloride concentration in the electrolysis bath after the input of the electrolysis accelerator by the electrolysis accelerator input unit. As a result, it is possible to grasp the sodium chloride concentration in the electrolysis bath after the input of the electrolysis accelerator by the electrolysis accelerator input unit.

Furthermore, the space purification device may further include a sodium chloride concentration addition unit that performs, a predetermined number of times, processing of adding a divided increase value to the sodium chloride concentration in the electrolysis bath every unit time after the input of the electrolysis accelerator by the electrolysis accelerator input unit, the divided increase value being obtained by dividing an increase theoretical value of the sodium chloride concentration due to the input of the electrolysis accelerator by the predetermined number of times, in which the unit time may be a time obtained by dividing a time until the sodium chloride concentration in the electrolysis bath is increased by the increase theoretical value by the predetermined number of times. This makes it possible to accurately grasp the sodium chloride concentration in the electrolysis bath after the input of the electrolysis accelerator by the electrolysis accelerator input unit.

Furthermore, the electrode controller may perform energization control to alternately perform energization and non-energization by the electrode unit, and the electrolysis accelerator input unit may be configured to wait until a non-energization state and to input the electrolysis accelerator in the non-energization state when a timing of inputting the electrolysis accelerator is in an energization state. This makes it possible to stably set the hypochlorous acid concentration in the electrolysis bath after completion of energization to the target hypochlorous acid concentration while prolonging the life of electrode unit 140.

Furthermore, the electrode controller may perform energization control in which energization and non-energization by the electrode unit are alternately performed, an energization direction is reversed from a first energization direction after a total energization time in the first energization direction has elapsed a second time, and a first non-energization time is provided after reverse of the energization direction, and the electrolysis accelerator input unit may be configured to input the electrolysis accelerator in a non-energization state. This makes it possible to stably set the hypochlorous acid concentration in the electrolysis bath after completion of energization to the target hypochlorous acid concentration while prolonging the life of electrode unit 140.

### INDUSTRIAL APPLICABILITY

The space purification device according to the present disclosure is useful as a space purification device that performs removal (including inactivation) of bacteria, fungi, viruses, odors, and the like in the air.

### REFERENCE MARKS IN THE DRAWINGS

100 electrolysis bath
110 water supply unit
112 lid
120 electrolytic water supply unit
122 water supply pump
124 water supply pipe
126 supply port
130 electrolysis bath full water float
140 electrode unit
200 purification bath
210 purification bath full water float
300 electrolysis accelerator input unit
310 electrolysis accelerator
400 purification unit
401 purification unit
500 controller
510 first calculator
512 second calculator
514 third calculator
516 fourth calculator
518 fifth calculator
520 sixth calculator
522 first change unit
524 seventh calculator
526 eighth calculator
528 second change unit
532 sodium chloride concentration addition unit
540 electrode controller
550 electrolytic water supply controller
560 electrolysis accelerator input controller
570 storage
600 controller
1000 space purification device
2000 space purification device

1. A space purification device comprising:
an electrolysis bath that mixes an electrolysis accelerator and water;
a water supply unit that supplies the water to the electrolysis bath;
an electrode unit that generates hypochlorous acid water from the electrolysis accelerator and the water mixed in the electrolysis bath;
a first calculator that calculates an increased concentration of hypochlorous acid water per unit time based on (i) a first sodium chloride concentration that is a sodium chloride concentration in the electrolysis bath after the sodium chloride concentration has changed due to an input of the electrolysis accelerator into the electrolysis bath and (ii) hypochlorous acid generation efficiency per unit time set in advance;
a second calculator that calculates, based on a target hypochlorous acid water concentration in the electrolysis bath, a first hypochlorous acid water concentration that is a hypochlorous acid water concentration in the electrolysis bath before energization by the electrode unit, and the increased concentration of hypochlorous acid water per unit time calculated by the first calculator, a required energization time for achieving the target hypochlorous acid water concentration; and
an electrode controller that performs energization in the electrode unit for the required energization time calculated by the second calculator.

2. The space purification device according to claim 1, further comprising a third calculator that calculates a second sodium chloride concentration that is the sodium chloride concentration in the electrolysis bath after the energization by the electrode unit based on the first sodium chloride concentration, the increased concentration of hypochlorous acid water per unit time, and the required energization time.

3. The space purification device according to claim 2, further comprising:
a purification bath that stores the hypochlorous acid water generated in the electrolysis bath;
an electrolytic water supply unit that supplies the hypochlorous acid water from the electrolysis bath to the purification bath; and
a purification unit that purifies a space using the hypochlorous acid water stored in the purification bath.

4. The space purification device according to claim 3, further comprising:
an electrolytic water supply controller that supplies electrolytic water to the purification bath by the electrolytic water supply unit;
a fourth calculator that calculates an amount of supplied electrolytic water by the electrolytic water supply unit; and
a fifth calculator that calculates a third sodium chloride concentration that is the sodium chloride concentration in the electrolysis bath after a supply of the electrolytic water by the electrolytic water supply unit based on the second sodium chloride concentration calculated by the third calculator, the amount of supplied electrolytic water calculated by the fourth calculator, and a capacity of the electrolysis bath.

5. The space purification device according to claim 4, further comprising a sixth calculator that calculates a second hypochlorous acid water concentration that is the hypochlorous acid water concentration in the electrolysis bath after the supply of the electrolytic water by the electrolytic water supply unit based on the target hypochlorous acid water concentration, the amount of supplied electrolytic water, and the capacity of the electrolysis bath.

6. The space purification device according to claim 5, further comprising a first change unit that changes the first sodium chloride concentration to the third sodium chloride concentration calculated by the fifth calculator and changes the first hypochlorous acid water concentration to the second hypochlorous acid water concentration calculated by the sixth calculator,
wherein the first calculator calculates a new increased concentration of hypochlorous acid water per unit time based on (i) a new first sodium chloride concentration changed by the first change unit and (ii) the hypochlorous acid generation efficiency per unit time set in advance,
the second calculator calculates a new required energization time by the electrode unit based on the target hypochlorous acid water concentration in the electrolysis bath, a new first hypochlorous acid water concentration changed by the first change unit, and the new increased concentration of hypochlorous acid water per unit time calculated by the first calculator, and
the electrode controller is configured to perform energization in the electrode unit for the new required energization time calculated by the second calculator.

7. The space purification device according to claim 6, wherein the space purification device is configured to repeat the change of the first sodium chloride concentration and the change of the first hypochlorous acid water concentration by the first change unit, the calculation of the new increased concentration of hypochlorous acid water per unit time by the first calculator, the calculation of the new required energization time by the second calculator, and the control of energization for the new required energization time by the electrode controller.

8. The space purification device according to claim 2, further comprising a purification unit that purifies a space using the hypochlorous acid water in the electrolysis bath.

9. The space purification device according to claim 8, further comprising:
a seventh calculator that calculates a hypochlorous acid decrease amount decreased from the electrolysis bath due to the purification by the purification unit; and
an eighth calculator that calculates a third hypochlorous acid water concentration that is the hypochlorous acid water concentration in the electrolysis bath after decrease of hypochlorous acid by the purification based on the target hypochlorous acid water concentration and the hypochlorous acid decrease amount calculated by the seventh calculator.

10. The space purification device according to claim 9, further comprising a second change unit that changes the first sodium chloride concentration to the second sodium chloride concentration calculated by the third calculator and changes the first hypochlorous acid water concentration to the third hypochlorous acid water concentration calculated by the eighth calculator,
wherein the first calculator calculates a new increased concentration of hypochlorous acid water per unit time based on (i) a new first sodium chloride concentration changed by the second change unit and (ii) the hypochlorous acid generation efficiency per unit time set in advance,
the second calculator calculates a new required energization time by the electrode unit based on the target hypochlorous acid water concentration in the electrolysis bath, a new first hypochlorous acid water concentration changed by the second change unit, and the new increased concentration of hypochlorous acid water per unit time calculated by the first calculator, and
the electrode controller is configured to perform energization in the electrode unit for the new required energization time calculated by the second calculator.

11. The space purification device according to claim 10, wherein the space purification device is configured to repeat the change of the first sodium chloride concentration and the change of the first hypochlorous acid water concentration by the second change unit, the calculation of the new increased concentration of hypochlorous acid water per unit time by the first calculator, the calculation of the new required energization time by the second calculator, and the control of energization for the new required energization time by the electrode controller.

12. The space purification device according to claim 1, further comprising an electrolysis accelerator input unit that inputs the electrolysis accelerator into the electrolysis bath,
wherein the electrolysis accelerator input unit is configured to input the electrolysis accelerator when the required energization time calculated by the second calculator is longer than a longest energization time.

13. The space purification device according to claim 3, further comprising an electrolysis accelerator input unit that inputs the electrolysis accelerator into the electrolysis bath,
wherein the electrolytic water supply controller supplies electrolytic water to the purification bath by the electrolytic water supply unit every first time, and
the electrolysis accelerator input unit is configured to input the electrolysis accelerator when the required energization time calculated by the second calculator is longer than the first time.

14. The space purification device according to claim 12, further comprising a sodium chloride concentration addition unit that adds an increase theoretical value of the sodium chloride concentration due to the input of the electrolysis accelerator to the sodium chloride concentration in the electrolysis bath after the input of the electrolysis accelerator by the electrolysis accelerator input unit.

15. The space purification device according to claim 12, further comprising a sodium chloride concentration addition unit that performs, a predetermined number of times, processing of adding a divided increase value to the sodium chloride concentration in the electrolysis bath every unit time after the input of the electrolysis accelerator by the electrolysis accelerator input unit, the divided increase value being obtained by dividing an increase theoretical value of the sodium chloride concentration due to the input of the electrolysis accelerator by the predetermined number of times,
wherein the unit time is a time obtained by dividing a time until the sodium chloride concentration in the electrolysis bath is increased by the increase theoretical value by the predetermined number of times.

16. The space purification device according to claim 12, wherein
the electrode controller performs energization control to alternately perform energization and non-energization by the electrode unit, and
the electrolysis accelerator input unit is configured to wait until a non-energization state and to input the electrolysis accelerator in the non-energization state when a timing of inputting the electrolysis accelerator is in an energization state.

17. The space purification device according to claim 12, wherein
the electrode controller performs energization control in which energization and non-energization by the electrode unit are alternately performed, an energization direction is reversed from a first energization direction after a total energization time in the first energization direction has elapsed a second time, and a first non-energization time is provided after reverse of the energization direction, and
the electrolysis accelerator input unit is configured to input the electrolysis accelerator in a non-energization state.
